(19) United States
(12) Patent Application Publication
(10) Pub. No.: US 2023/0084402 A1
(43) Pub. Date: Mar. 16, 2023

(54) Biomarkers for Risk Prediction of Parkinson's Disease

(71) Applicants: AGENCY FOR SCIENCE, TECHNOLOGY AND RESEARCH, Singapore (SG); SINGAPORE HEALTH SERVICES PTE LTD, Singapore (SG)

(72) Inventors: Jia Nee Foo, Singapore (SG); Jianjun Liu, Singapore (SG); Eng-King Tan, Singapore (SG)

(21) Appl. No.: 17/796,293

(22) PCT Filed: Feb. 5, 2021
(86) PCT No.: PCT/SG2021/050063

§ 371 Date: Jul. 29, 2022

(30) Foreign Application Priority Data
Feb. 5, 2020 (SG) 10202001048U

(51) Int. Cl. C12Q 1/6883

(52) U.S. Cl. CPC C12Q 1/6883; C12Q 2600/156

(57) ABSTRACT

The present invention refers to a method of identifying whether a subject is at risk of developing PD (PD), whether a subject is suffering from PD, or whether a subject is in need of early therapeutic intervention for PD, the method comprising detecting the presence of a genetic variant at the loci of one or more genes selected from the group consisting of SV2C, WBSCR17, PARK16, ITPKB, MCCC1, SNCA, FAM47E-SCARB2, FYN, DLG2, LRRK2, RIT2 and combinations thereof in a sample obtained from the subject, wherein the presence of one or more genetic variants identifies that the subject is at risk of developing PD, the subject is suffering from PD, or the subject is in need of early therapeutic intervention for PD. Also, described herein are a method of determining the prognosis of a subject with PD or a subject at risk of developing PD and a method for calculating a polygenic risk score (PRS) of a subject of developing PD. Further, described herein are biomarkers and kits for PD.

### CROSS-REFERENCE TO RELATED APPLICATIONS

This application claims the benefit of priority of Singapore application No. 10202001048U, filed 5 Feb. 2020, the contents of it being hereby incorporated by reference in its entirety for all purposes.

### FIELD OF THE INVENTION

The invention is in the field of biomarkers, in particular biomarkers associated with Parkinson's disease and methods and uses thereof.

### BACKGROUND OF THE INVENTION

Parkinson's disease (PD) is one of the most common age-related neurodegenerative diseases worldwide and has contributed to over 200,000 deaths and 3.2 million disability-adjusted life years worldwide in 2016. PD presents as a hypokinetic movement disorder characterized by bradykinesia, postural instability, rigidity and resting tremors resulting from loss of nigrostriatal dopaminergic neurons and other non-dopaminergic structures. At present, there is no cure for PD as symptoms only present at late stages of the disease. Several genes containing rare pathogenic variants have been identified in familial PD, suggesting that while genetic factors play a role in PD pathogenesis, it is extremely heterogeneous and influenced by multiple genes and pathways. It implies that germ line genetic variants may serve as stable biomarkers for risk prediction early in life. Despite the large-scale meta-analyses of genome-wide association studies (GWAS) in the European population having identified several dozen loci with implication in PD pathogenesis and confirmed the involvement of familial PD genes in sporadic PD, there are limited studies in the Asian population which is the largest worldwide, and thus makes up a significant fraction of PD patients globally.

It is therefore important to identify biomarkers that can be used to diagnose PD, predict risk and identify at-risk individuals for early monitoring and therapeutic intervention. In addition, there is also a need to identify novel, potentially Asian-specific biomarkers to conduct a robust comparison between Asian and European genetic risk for PD.

### SUMMARY

In one aspect, there is provided a method of identifying whether a subject is at risk of developing PD, whether a subject is suffering from PD, or whether a subject is in need of early therapeutic intervention for PD, the method comprising: a. obtaining a DNA sample from the subject; and b. detecting the presence of a genetic variant at the loci of one or more genes selected from the group consisting of SV2C, WBSCR17, PARK16, ITPKB, MCCC1, SNCA, FAM47E-SCARB2, FYN, DLG2, LRRK2, RIT2 and combinations thereof in the sample; wherein the presence of one or more genetic variants identifies that the subject is at risk of developing PD, the subject is suffering from PD, or the subject is in need of early therapeutic intervention for PD.

In one aspect, there is provided a method of determining the prognosis of a subject with PD or a subject at risk of developing PD, the method comprising: a. obtaining a DNA sample from the subject; and b. detecting the presence of a genetic variant at the loci of one or more genes selected from the group consisting of SV2C, WBSCR17, PARK16, ITPKB, MCCC1, SNCA, FAM47E-SCARB2, FYN, DLG2, LRRK2, RIT2 and combinations thereof in the sample; wherein the presence of one or more genetic variants indicates that the subject has a poor prognosis.

In another aspect, there is provided a method of calculating a polygenic risk score (PRS) of a subject of developing PD, the method comprising the steps of: a. obtaining a DNA sample from the subject; b. detecting the presence of a genetic variant at the loci of one or more genes selected from the group consisting of SV2C, WBSCR17, PARK16, ITPKB, MCCC1, SNCA, FAM47E-SCARB2, FYN, DLG2, LRRK2, RIT2 and combinations thereof in the sample; and running genotyping analysis of DNA; and c. measuring the total number of the genetic variants detected in step b to calculate a PRS of a subject of developing PD.

In another aspect, there is provided a kit comprising one or more reagents to detect the presence of a genetic variant at the loci of one or more genes selected from the group consisting of SV2C, WBSCR17, PARK16, ITPKB, MCCC1, SNCA, FAM47E-SCARB2, FYN, DLG2, LRRK2, RIT2 and combinations thereof in a sample, together with instructions for use.

In yet another aspect, there is provided a PD biomarker, wherein the biomarker is a genetic variant at the loci of one or more genes selected from the group consisting of SV2C, WBSCR17, PARK16, ITPKB, MCCC1, SNCA, FAM47E-SCARB2, FYN, DLG2, LRRK2, RIT2 and combinations thereof.

### DEFINITIONS

The following are some definitions that may be helpful in understanding the description of the present invention. These are intended as general definitions and should in no way limit the scope of the present invention to those terms alone, but are put forth for a better understanding of the following description.

As used herein, the term “prognosis” refers to a prediction of the probable course and outcome of a clinical condition or disease. The prognosis, as used herein, can also refer to requirement of therapeutic intervention according to the course and outcome of a clinical condition or disease. A prognosis of a patient is usually made by evaluating factors or symptoms of a disease that are indicative of a favorable or unfavorable course or outcome of the disease. The term “prognosis” does not refer to the ability to predict the course or outcome of a condition with 100% accuracy. Instead, the term “prognosis” refers to an increased probability that a certain course or outcome will occur; that is, that a course or outcome is more likely to occur in a patient exhibiting a given condition, when compared to those individuals not exhibiting the condition. For example, the course or outcome of a condition may be predicted with 99%, 98%, 97%, 96%, 95%, 94%, 93%, 92%, 91%, 90%, 89%, 88%, 87%, 86%, 85%, 84%, 83%, 82%, 81%, 80%, 75%, 70%, 65%, 55%, and 50% accuracy.

As used herein, the term “biomarker” refers to a molecular indicator of a specific biological property, a biochemical feature or facet that can be used to determine the presence or absence and/or severity of a particular disease or condition. One or more biomarkers may be associated with the particular disease or condition. The term “biomarker” may refer to a polypeptide or nucleic acid sequence encoding the polypeptide, a fragment or variant of the polypeptide that is associated with PD. In addition, a “biomarker” can also refer to metabolites or metabolized fragments of the expressed polypeptide. A person skilled in the art would understand that a metabolite of one of the biomarkers referred to herein can still retain the capability of being used as biomarker for the methods described herein. It is also noted that some of the biomarkers in the biomarker set can be present in their variant form or metabolized form while others are still intact. In the present disclosure, the term “biomarker” refers to, but is not limited to, one or more genetic variants, a sequence encoding the genetic variant, the resulting mRNA, or the resulting polypeptide or protein if the genetic variation affects the protein-coding region. For example, a biomarker may be a combination of genetic variants at the loci of one or more genes. Evaluation of such biomarkers and their correlation to a pathological condition or disease can be done by, for example, determining the absence or presence of a biomarker, and comparative analysis between diseased and disease-free samples.

As used herein, the term “polymorphism” refers genetic polymorphism, which is used to describe diversity in genomes in species, such as a human being. It essentially refers to inter-individual differences in a DNA sequence that is unique to an individual. In other words, a genetic polymorphism is the occurrence, in the same population, of multiple discrete allelic states. Polymorphism involves one of two or more variants of a particular DNA sequence. The most common type of polymorphism involves variation at a single nucleotide, i.e., single nucleotide polymorphism (SNP).

As used herein, the terms “variant” or “genetic variant” refer to a specific region of the genome that differs from a reference genome. Based on the type of alteration, the term “genetic variant” can refer to, but is not limited to, single nucleotide variant (SNV) or single nucleotide polymorphism (SNP). As used herein, the term “SNV” or “SNP” refers to a variant with a single nucleotide substitution in a DNA sequence. Conventionally a SNP is a SNV that is present to some appreciable degree within a population (for example, more than 1% of said population).

SNPs may occur in all positions of the DNA sequence encoding the genetic variant, such as coding regions, non-coding regions, or the regions between genes. They can occur, for example, in the exons, introns, UTRs, regulatory regions such as enhancer, transcription factor binding domain and DNA methylation regions or regions with no known function.

As used herein, the term “locus” refers to a specific position on a chromosome. It is known that multiple genes can reside at the same locus. It would be understood by a person skilled in the art that a SNP occurs at a specific locus on the chromosome which can be either within a gene or in the region between two genes. The locus where a SNP occurs may be named according to the gene that is nearest to the SNP. For example, the locus where SNP rs34311866 occurs may be named as “GAK”. The locus where a SNP occurs may be also named according to multiple genes that are located at varying distances from the SNP within the locus. For example, the locus where SNP rs34311866 occurs may also be named as “TMEM175-GAK-DGKQ”.

As used herein, the term “polygenic score” or “polygenic risk score (PRS)” is a score based on the variation in multiple genetic loci and their associated weights. The PRS is constructed from the effect size for each risk allele or effect allele and generally follows the form:

$$\hat{S} = {\sum }_{j = 1}^{m} {X}_{j} {\hat{β}}_{j}$$

where the PRS, Ŝ of an individual is equal to the weighted sum of the individual's marker genotypes, Xⱼ, at m genetic variants or small nucleotide polymorphisms (SNPs). Weights \hat{β}ⱼ are estimated using regression analysis, such as logistic regression.

As used herein, the term “principal component analysis (PCA)” refers to a statistical procedure that uses an orthogonal transformation to convert a set of observations of possibly correlated variables into a set of values of linearly uncorrelated variables called principal components. PCA may be used to detect and correct allele frequency differences between an individual and controls (one or more individuals of know ancestry) due to systemic ancestry differences, thereby allowing ancestry differences between an individual and controls to be modelled.

As used herein, the terms “isolated” or “isolating” relates to a biological component (such as a nucleic acid molecule, protein or organelle) that has been substantially separated or purified away from other biological components in the cell of the organism in which the component naturally occurs, i.e., other chromosomal and extra-chromosomal DNA and RNA, proteins and organelles. Nucleic acids that have been “isolated” include nucleic acids purified by standard purification methods.

As used herein, the term “sample”, refers to single cells, multiple cells, fragments of cells, tissue, or body fluid, which has been obtained from, removed from, or isolated from a subject. An example of a sample includes, but is not limited to, blood, stool, serum, plasma, tears, saliva, urine, sputum, nasal fluid, gastrointestinal fluid, cerebrospinal fluid, bone marrow fluid, exudate, transudate, bronchial lavage. In another example, the biomarker may be fresh tissue, frozen fresh tissue, paraffin embedded tissue or formalin fixed paraffin embedded tissue. The sample can include, but is not limited to, tissue obtained from the brain, lung, muscle, brain, liver, skin, pancreas, stomach, bladder, and other organs.

As used herein, the term “primer” refers to any single-stranded oligonucleotide sequence capable of being used as a primer in, for example, PCR technology. Thus, a “primer” according to the disclosure refers to a single-stranded oligonucleotide sequence that is capable of acting as appoint of initiation for synthesis of a primer extension product that is substantially identical to the nucleic acid strand to be copied (for a forward primer) or substantially the reverse complement of the nucleic acid strand to be copied (for a reverse primer).

As used herein, the term “probe” refers to any nucleic acid fragment that hybridizes to a target sequence. A probe may be labelled with radioactive isotopes, fluorescent tags, antibodies or chemical labels to facilitate detection of the probe.

### BRIEF DESCRIPTION OF THE DRAWINGS

The invention will be better understood with reference to the detailed description when considered in conjunction with the non-limiting examples and the accompanying drawings, in which:

FIG. 1 Genome-wide association study of East Asian PD. Manhattan plot from meta-GWAS of five East Asian sample collections, with novel loci (with arrowhead) and previously-reported loci (without arrowhead). Genome-wide significant loci are indicated in underline font.

FIG. 2 Two novel PD risk loci. (A, C) Recombination and (B, D) forest plots showing associations at (A, B) SV2C and (C, D) WBSCR17 in the Asian meta-GWAS. (A) Recombination showing association at SV2C. (B) Forest plot showing association at SV2C. (C) Recombination showing association at WBSCR17. (D) Forest plot showing association at WBSCR17.

FIG. 3 PRS analysis in Asian samples. (A) PRS distribution using 11 genome-wide significant Asian SNPs. (B) 90 known PD SNPs (78 polymorphic) identified in European samples. (C) Receiver operator curve (ROC) based on polygenic risk prediction of PD with previously-reported SNPs (solid line) vs combined European and Asian SNPs (dotted line).

### DETAILED DESCRIPTION OF THE PRESENT INVENTION

In one aspect, the present invention refers to a method of identifying whether a subject is at risk of developing PD, whether a subject is suffering from PD, or whether a subject is in need of early therapeutic intervention for PD, the method comprising: a) obtaining a DNA sample from the subject; and b) detecting the presence of a genetic variant at the loci of one or more genes selected from the group consisting of SV2C, WBSCR17, PARK16, ITPKB, MCCC1, SNCA, FAM47E-SCARB2, FYN, DLG2, LRRK2, RIT2 and combinations thereof in the sample; wherein the presence of one or more genetic variants identifies that the subject is at risk of developing PD, the subject is suffering from PD, or the subject is in need of early therapeutic intervention for PD.

In one example, the method involves detecting the presence of a genetic variant at the loci of SV2C and WBSCR17.

In another example, the method involves detecting the presence of a genetic variant at the loci of SV2C, WBSCR17, PARK16, ITPKB, MCCC1, SNCA, FAM47E-SCARB2, FYN, DLG2, LRRK2 and RIT2. Full name of the 11 genetic loci can be found in Table 1.

TABLE 1
Full name of the 11 genetic loci

| Genetic locus | Full name of the genetic locus | Also known as |
|---|---|---|
| SV2C | synaptic vesicle glycoprotein 2C | |
| WBSCR17 | polypeptide N-acetylgalactosaminyl- | GALNT17 |
| | transferase 17 | |
| PARK16 | Parkinson disease 16 (susceptibility) | |
| ITPKB | leucine rich repeat kinase 2 | |
| MCCC1 | methylcrotonoyl-CoA carboxylase 1 | |
| SNCA | synuclein alpha | |
| FAM47E- | family with sequence similarity 47 | |
| SCARB2 | member E-scavenger receptor class B | |
| | member 2 | |
| FYN | FYN proto-oncogene, Src family | |
| | tyrosine kinase | |
| DLG2 | discs large MAGUK scaffold protein 2 | |
| LRRK2 | leucine rich repeat kinase 2 | |
| RIT2 | Ras like without CAAX 2 | |

The method of the invention can therefore be used either identify whether a subject is at risk of developing PD, or whether a subject is suffering from PD.

A subject or patient who is suffering from PD either has already been diagnosed with, or has not yet been diagnosed with PD. The subject or patient may be symptomatically characterized by one or more of the following features, but not limited to, bradykinesia, postural instability, rigidity, resting tremors, loss of automatic movements, changes in speech and writing, and cognitive impairment. The subject may also be patho-physiologically characterized by one or more of the following features, but not limited to, loss of nigrostriatal dopaminergic neurons and other non-dopaminergic structures. In one example, the characteristics of PD is assessed using the United Kingdom Parkinson's Society Brain Bank Criteria.

A subject or patient who is at risk of developing PD has a higher likelihood of developing PD relative to the rest of the population. The higher likelihood may be attributed to factors including, but not limited to, genetic variations and environmental triggers such as exposure to certain toxins. In some example, the higher risk is due to genetic predisposition or susceptibility. A subject or patient is said to be developing PD or have developed PD based on the manifestation of symptoms of PD, such as bradykinesia, postural instability, rigidity, resting tremors, loss of automatic movements, changes in speech and writing, and cognitive impairment, and/or pathological characteristics, such as loss of nigrostriatal dopaminergic neurons and other non-dopaminergic structures.

A subject who is identified as being at risk of developing PD may or may not also be in need of early therapeutic intervention. Similarly, a person who is suffering from PD may or may not also be in need of early therapeutic intervention. Therefore, provided here is also a method to identify whether a subject is in need of early therapeutic intervention for PD.

In one example, early therapeutic intervention includes but is not limited to one or more of the following: monitoring the subject for disease onset and progression, prophylactic treatment with a neuroprotective drug, and dietary or lifestyle changes.

As part of early therapeutic intervention, the subject may be monitored regularly for the onset of PD and/or progression. Further therapeutic intervention may be prescribed based on the outcome of the monitoring.

Early therapeutic intervention may also include prophylactic treatment. Prophylactic treatment in the context of PD refers to a treatment or intervention that is designed and used to prevent PD disease from occurring, to delay the onset of PD, to reduce the severity of PD or combinations thereof. For example, a prophylactic treatment for PD can be a neuroprotective drug that is commercially available or in clinical trials. It will generally be understood that a neuroprotective drug or a neuroprotective agent is a compound or agent that is capable of salvaging, recovering and/or regenerating the nervous system, neural cells, neural structure or neural function.

Other early intervention therapies include dietary or lifestyle changes such as changes to diet, nutrition intake and exercise.

A genetic variant can occur in many forms, which include, but are not limited to, SNV or SNP. In one example, a genetic variant refers to a SNP.

The genetic variant may be detected in any position of the DNA sequence encoding the genetic variant, for example, exons, introns, UTRs, other regulatory regions or regions without known functions. For example, the genetic variant may be a SNP detected within an intron of a gene.

The consequence of the genetic variation can be synonymous or non-synonymous. For example, the genetic variant may be a synonymous or non-synonymous SNP that occurs in the exon of the gene. Synonymous SNPs are those SNPs that have different alleles that encode for the same amino acid. Non-synonymous SNPs are SNPs that have different alleles that encode different amino acids. A synonymous variant occurs when the nucleotide substitution does not result in a change in amino acid, while a non-synonymous variant occurs when the nucleotide substitution leads to an amino acid substitution. In some example, the non-synonymous SNPs may be missense, nonsense or frameshift. Missense refers to where the nucleotide substitution results in a codon that codes for a different amino acid. Nonsense refers to where the nucleotide substitution results in a premature stop codon and truncation of protein. For example, a non-synonymous SNP may be a missense variant.

A subject who has been identified as having or suffering from PD, or as being at risk of developing PD may also be tested to determine their prognosis. As such, in another aspect, the present invention refers to a method of determining the prognosis of a subject with PD or a subject at risk of developing PD, the method comprising: a). obtaining a DNA sample from the subject; and b). detecting the presence of a genetic variant at the loci of one or more genes selected from the group consisting of SV2C, WBSCR17, PARK16, ITPKB, MCCC1, SNCA, FAM47E-SCARB2, FYN, DLG2, LRRK2, RIT2 and combinations thereof in the sample; wherein the presence of one or more genetic variants indicates that the subject has a poor prognosis.

The prognosis of a subject in the context of PD includes but is not limited to the response of a subject to a treatment for PD, the progression of PD, the age of onset of PD, the need for early and/or aggressive therapy for PD. A poor prognosis therefore may mean that a subject is not responsive or not likely to respond to PD treatment. A poor prognosis may also mean that a subject is likely to have a rapid progression of PD or a rapid onset of symptoms associated with PD. Further, a poor prognosis may mean that the onset of PD happened or is likely to happen at an early or earlier age relative to a subject that has a good prognosis. A subject with a poor prognosis of PD may also require early and/or aggressive therapy for PD.

Early therapy refers to the treatment of a subject at an early stage of PD. For example, where the symptoms of PD are mild. Aggressive PD therapy refers to the treatment of a subject with more types of drugs, higher doses of drugs, higher frequency of treatment or more types of treatments. Aggressive PD therapy may also refer to intensive monitoring of high risk individuals at pre-symptomatic stage or early stages, and possible participation in trials for neuroprotective therapy.

In one example, the method involves detecting the presence of a genetic variant at the loci of SV2C and WBSCR17.

In another example, the method involves detecting the presence of a genetic variant at the loci of SV2C, WBSCR17, PARK16, ITPKB, MCCC1, SNCA, FAM47E-SCARB2, FYN, DLG2, LRRK2 and RIT2.

In addition to detecting genetic variants at the loci of one or more genes described in the foregoing, the method may further detect the presence of genetic variants at the loci of one or more additional genes. In one example, the one or more additional genes is selected from the group consisting of ILIR2, SCN3A, SATB1, NCKIPSD, CDC71, ALAS1, TLR9, DNAH1, BAP1, PHF7, NISCH, STAB I, ITIH3, ITIH4, ANK2, CAMK2D, ELOVL7, ZNF184, CTSB, SORBS3, PDLIM2, C8orf58, BIN3, SH3GL2, FAM171A1, GALC, COQ7, TOX3, ATP6V0A1, PSMC3I, TUBG2, GBA-SYT11, RAB7L1-NUCKS1, SIPA1L2, ACMSD-TMEM163, STK39, KRT8P25-APOOP2, NMD3, TMEM175-GAK-DGKQ, BST1, HLA-DQB1, GPNMB, FGF20, MMP16, ITGA8, INPP5F, MIR4697, LRRK2, CCDC62, GCH1, TMEM229B, VPS13C, BCKDK-STXIB, SREBF1-RAI1, MAPT, SPPL2B, DDRGKI, USP25, FCGR2A, VAMP4, KCNS3, KCNIP3, LINC00693, KPNA1, MED12L, SPTSSB, LCORL, CLCN3, PAM, C5orf24, TRIM40, RIMS1, RPS12, GS1-124K5.11, FAM49B, UBAP2, GBF1, RNF141, SCAF11, FBRSLI, CAB39L, MBNL2, MIPOL1, RPS6KL1, CD19, NOD2, CNOT1, CHRNBI, UBTF, FAM171A2, BRIP1, DNAH17, ASXL3, MEX3C, CRLS1, DYRKIA and combinations thereof.

In one example, in addition to detecting a genetic variant at the loci of SV2C, WBSCR17, PARK16, ITPKB, MCCC1, SNCA, FAM47E-SCARB2, FYN, DLG2, LRRK2 and RIT2, a genetic variant is further detected at the loci of BST1, GAK, ASXL3, VPS13C, FGF20, RPS12, ZNF184, SH3GL2, CCDC62, LCORL, RIMS1, UBAP2, RNF141, SCAF11, FBRSLI, RPS6KL1, UBTF and STK39.

In another example, in addition to detecting a genetic variant at the loci of SV2C, WBSCR17, PARK16, ITPKB, MCCC1, SNCA, FAM47E-SCARB2, FYN, DLG2, LRRK2 and RIT2, a genetic variant is further detected at the loci of ILIR2, SCN3A, SATB1, NCKIPSD, CDC71, ALAS1, TLR9, DNAH1, BAP1, PHF7, NISCH, STAB I, ITIH3, ITIH4, ANK2, CAMK2D, ELOVL7, ZNF184, CTSB, SORBS3, PDLIM2, C8orf58, BIN3, SH3GL2, FAM171A1, GALC, COQ7, TOX3, ATP6V0A1, PSMC3I, TUBG2, GBA-SYT11, RAB7L1-NUCKS1, SIPA1L2, ACMSD-TMEM163, STK39, KRT8P25-APOOP2, NMD3, TMEM175-GAK-DGKQ, BST1, HLA-DQB1, GPNMB, FGF20, MMP16, ITGA8, INPP5F, M1R4697, LRRK2, CCDC62, GCH1, TMEM229B, VPS13C, BCKDK-STXIB, SREBF1-RAI1, MAPT, SPPL2B, DDRGKI, USP25, FCGR2A, VAMP4, KCNS3, KCNIP3, L1NC00693, KPNA1, MED12L, SPTSSB, LCORL, CLCN3, PAM, C5orf24, TRIM40, RIMS1, RPS12, GS1-124K5.11, FAM49B, UBAP2, GBF1, RNF141, SCAF11, FBRSLI, CAB39L, MBNL2, MIPOLI, RPS6KL1, CD19, NOD2, CNOT1, CHRNBI, UBTF, FAM171A2, BRIP1, DNAH17, ASXL3, MEX3C, CRLS1 and DYRKIA.

The present invention also provides a method of calculating a risk score for the likelihood or risk of a subject developing PD. In one aspect, the present invention refers to a method of calculating a PRS of a subject of developing PD, the method comprising the steps of: a. obtaining a DNA sample from the subject; b. detecting the presence of a genetic variant at the loci of one or more genes selected from the group consisting of SV2C, WBSCR17, PARK16, ITPKB, MCCC1, SNCA, FAM47E-SCARB2, FYN, DLG2, LRRK2, RIT2 and combinations thereof in the sample; and c. measuring the total number of the genetic variants detected in step b to calculate a PRS of a subject of developing PD.

In one example, the method of calculating a PRS involves detecting the presence of a genetic variant and measuring the total number of genetic variants at the loci of SV2C and WBSCR17.

In another example, the method of calculating a PRS involves detecting the presence of a genetic variant and measuring the total number of genetic variants at the loci of SV2C, WBSCR17, PARK16, ITPKB, MCCC1, SNCA, FAM47E-SCARB2, FYN, DLG2, LRRK2 and RIT2.

In addition to detecting the presence of a genetic variant and measuring the total number of genetic variants at the loci of SV2C, WBSCR17, PARK16, ITPKB, MCCC1, SNCA, FAM47E-SCARB2, FYN, DLG2, LRRK2 and RIT2 genes, the method of calculating a PRS may further comprise detecting the presence of a genetic variant and measuring the total number of genetic variants at the loci of one or more additional genes. In one example, the one or more additional genes is selected from the group consisting of ILIR2, SCN3A, SATB1, NCKIPSD, CDC71, ALAS1, TLR9, DNAH1, BAP1, PHF7, NISCH, STAB1, ITIH3, ITIH4, ANK2, CAMK2D, ELOVL7, ZNF184, CTSB, SORBS3, PDLIM2, C8orf58, BIN3, SH3GL2, FAM171A1, GALC, COQ7, TOX3, ATP6V0A1, PSMC3I, TUBG2, GBA-SYT11, RAB7L1-NUCKS1, SIPA1L2, ACMSD-TMEM163, STK39, KRT8P25-APOOP2, NMD3, TMEM175-GAK-DGKQ, BST1, HLA-DQB1, GPNMB, FGF20, MMP16, ITGA8, INPP5F, M1R4697, LRRK2, CCDC62, GCH1, TMEM229B, VPS13C, BCKDK-STXIB, SREBF1-RAI1, MAPT, SPPL2B, DDRGKI, USP25, FCGR2A, VAMP4, KCNS3, KCNIP3, L1NC00693, KPNA1, MED12L, SPTSSB, LCORL, CLCN3, PAM, C5orf24, TRIM40, RIMS1, RPS12, GS1-124K5.11, FAM49B, UBAP2, GBF1, RNF141, SCAF11, FBRSLI, CAB39L, MBNL2, MIPOLI, RPS6KL1, CD19, NOD2, CNOT1, CHRNBI, UBTF, FAM171A2, BRIP1, DNAH17, ASXL3, MEX3C, CRLS1, DYRKIA and combinations thereof.

In one example, the method of calculating a PRS comprises detecting the presence of a genetic variant and measuring the total number of genetic variants at the loci of the SV2C, WBSCR17, PARK16, ITPKB, MCCC1, SNCA, FAM47E-SCARB2, FYN, DLG2, LRRK2, RIT2, BST1, GAK, ASXL3, VPS13C, FGF20, RPS12, ZNF184, SH3GL2, CCDC62, LCORL, RIMS1, UBAP2, RNF141, SCAF11, FBRSL1, RPS6KL1, UBTF and STK39 genes.

In another example, the method of calculating a PRS comprises detecting the presence of a genetic variant and measuring the total number of genetic variants at the loci of the SV2C, WBSCR17, PARK16, ITPKB, MCCC1, SNCA, FAM47E-SCARB2, FYN, DLG2, LRRK2, RIT2, ILIR2, SCN3A, SATB1, NCKIPSD, CDC71, ALAS1, TLR9, DNAH1, BAP1, PHF7, NISCH, STAB1, ITIH3, ITIH4, ANK2, CAMK2D, ELOVL7, ZNF184, CTSB, SORBS3, PDLIM2, C8orf58, BIN3, SH3GL2, FAM171A1, GALC, COQ7, TOX3, ATP6V0A1, PSMC3I, TUBG2, GBA-SYT11, RAB7L1-NUCKS1, SIPA1L2, ACMSD-TMEM163, STK39, KRT8P25-APOOP2, NMD3, TMEM175-GAK-DGKQ, BST1, HLA-DQB1, GPNMB, FGF20, MMP16, ITGA8, INPP5F, MIR4697, LRRK2, CCDC62, GCH1, TMEM229B, VPS13C, BCKDK-STXIB, SREBF1-RAI1, MAPT, SPPL2B, DDRGKI, USP25, FCGR2A, VAMP4, KCNS3, KCNIP3, LINC00693, KPNA1, MED12L, SPTSSB, LCORL, CLCN3, PAM, C5orf24, TRIM40, RIMS1, RPS12, GS1-124K5.11, FAM49B, UBAP2, GBF1, RNF141, SCAF11, FBRSLI, CAB39L, MBNL2, MIPOL1, RPS6KL1, CD19, NOD2, CNOT1, CHRNBI, UBTF, FAM171A2, BRIP1, DNAH17, ASXL3, MEX3C, CRLS1 and DYRK1 genes.

In the method for calculating a PRS, the total number of genetic variants may be unweighted or weighted. In one example, the total number of genetic variants may be weighted by the effect size of each variant.

Effect size or beta (β) is a measure of how the risk of developing PD changes for every copy of risk allele or effect allele carried by an individual. It will generally be understood that each individual carries 2 copies of each chromosome (a paternal and a maternal chromosome) and can therefore carry either 0, 1 or 2 copies of a risk allele or effect allele. The “effect size” measures the relative risk of an individual carrying 2 copies of the risk allele versus 1 copy of the risk allele, or 1 copy of the risk allele versus 0 copies of the risk allele. By comparing the number of copies of a risk allele between patients suffering from PD and controls, an effect size for each risk allele or genetic variant can be determined. The effect size may also be expressed as an “odds ratio (OR)”, which is calculated by taking the exponential of the effect size or beta (β).

In one example, effect size may be −0.300, −0.200, −0.150, −0.100, −0.050, 0.050, 0.100, 0.150, 0.200, 0.250, 0.300, 0.350, 0.400, 0.500, 0.600, 0.700, 0.800 or 0.900. In one example, the reported effect size is 0.211. In another example, the reported effect size is 0.217. In yet another example, the reported effect size is 0.128.

In one example, the effect size is determined using logistic regression comparing genotypes in patients suffering from PD versus controls (patients who are not suffering from PD). The effect size is calculated for each risk allele or effect allele and combined to construct a PRS.

In one example, in the method for calculating a PRS of a subject of developing PD, the PRS of the subject is compared with PRSs in a reference population to determine the percentile risk of the subject's risk of developing PD. An example of reference population is a population without PD. Another example is a representative population of the general population whose PD status is unknown.

In one example, the PRS percentiles are used to estimate the fold-difference in risk of developing PD. In one example, PRS cut-offs for the top and bottom 5% are determined based on the control population, and number of PD disease cases in the first group with PRS higher than or equals to the top 5 percentile and in the second group with PRS lower than or equals to the bottom 5 percentile are then determined respectively to estimate the fold-difference in risk between the two groups in the disease population. In another example, PRS cut-offs for the top and bottom 10% are determined based on the control population, and number of PD disease cases in the first group with PRS higher than or equals to the top 10 percentile and in the second group with PRS lower than or equals to the bottom 10 percentile are then determined respectively to estimate the fold-difference in risk between the two groups in the disease population.

In one example, the PRS percentile is used to predict the risk of developing PD. In one example, a subject with a PRS that is in a higher percentile has a higher risk of developing PD compared to an individual with a PRS that is in a lower percentile. In another example, an individual with a lower percentile PRS has a lower risk of developing PD compared to an individual with a higher percentile PRS. It will therefore be understood that a subject with a PRS that is in the bottom 5 percentile has lowest risk of developing PD, and a subject with a PRS that is in the 95-100 percentile or the top 5 percentile has the highest risk of developing PD.

In another example, the PRS may be used to determine the prognosis of subject with PD, where a subject with a PRS in a higher percentile has a higher risk of having poor prognosis compared to a subject with a PRS that is in a lower percentile. Similarly, a subject with a PRS in lower percentile has a lower risk of poor prognosis compared to a subject with a PRS that is in a higher percentile.

In one example, in the method of identifying whether a subject is suffering from PD, at risk of developing PD, identifying whether a subject is in need of early therapeutic intervention for PD, determining the prognosis, or calculating a PRS of a subject of developing PD, the one or more genetic variants is a polymorphism.

In one example, the polymorphism is a SNV or SNP. For example, the genetic variant is an effect allele or risk allele of the SNP or SNV.

An effect allele refers to the allele whose effects in relation to the disease are being studied. In some examples, the effect allele may be the risk allele, which is the allele of a SNP that confers the risk of developing the disease. Such an allele has genome-wide significance and has an odds ratio >1.0, which indicates an increased risk relative to the other allele. In other words, risk allele is associated with a positive effect size as opposed to negative effect size. In the present disclosure, the term “effect allele” refers to the risk allele, which is confers the increased risk of developing PD.

In one example, the genetic variant is a SNP selected from the group consisting of rs6826785, rs141336855, rs6679073, rs2292056, rs16846351, rs3816248, rs12278023, rs9638616, rs1887316, rs246814, rs31244, rs4130047 and combinations thereof.

In one example, the genetic variants for the genes WBSCR17 and SV2C are rs9638616 and rs246814 respectively. In another example, the genetic variants for the genes WBSCR17 and SV2C are rs9638616 and rs31244 respectively.

In one example, the genetic variants are rs6826785, rs141336855, rs6679073, rs2292056, rs16846351, rs3816248, rs12278023, rs9638616, rs1887316, rs246814 and rs4130047. In another example, the genetic variants are rs6826785, rs141336855, rs6679073, rs2292056, rs16846351, rs3816248, rs12278023, rs9638616, rs1887316, rs31244 and rs4130047.

It is well understood that each reference SNP (rs) number can be used as an identification number for a specific SNP at the locus of a gene. In one example, rs246814 is a SNP located within an intron of the SV2C gene. In another example, rs31244 is a missense SNP located within SV2C. In yet another example, rs9638616 is a SNP located within an intron of the WBSCR17 gene.

In some examples, the genetic variant at the loci of SNCA is rs6826785, and the effect allele of rs6826785 is cytosine (C). In some examples, the genetic variant at the loci of LRRK2 is rs141336855, and the effect allele of rs141336855 is thymine (T). In some examples, the genetic variant at the loci of PARK16 is rs6679073, and the effect allele of rs6679073 is adenine (A). In some examples, the genetic variant at the loci of MCCCI is rs2292056, and the effect allele of rs2292056 is guanine (G). In some examples, the genetic variant at the loci of ITPKB is rs16846351, and the effect allele of rs16846351is guanine (G). In some examples, the genetic variant at the loci of FAM47E-SCARB2 is rs3816248, and the effect allele of rs3816248 is cytosine (C). In some examples, the genetic variant at the loci of DLG2 is rs12278023, and the effect allele of rs12278023 is cytosine (C). In some examples, the genetic variant at the loci of WBSCR17 is rs9638616, and the effect allele of rs9638616 is thymine (T). In some examples, the genetic variant at the loci of FYN is rs1887316, and the effect allele of rs1887316 is adenine (A). In some examples, the genetic variant at the loci of SV2C is rs246814 or rs31244, and the effect allele of rs246814 is thymine (T) and the effect allele of rs31244 is guanine (G). In some examples, the genetic variant at the loci of RIT2 is rs4130047, and the effect allele of rs4130047 is cytosine (C).

In another example, in addition to the genetic variants detected in the foregoing gene list, the method further comprises detecting the presence or measuring the total number of genetic variants at the loci of one or more genes selected from the group consisting of ILIR2, SCN3A, SATB1, NCKIPSD, CDC71, ALAS1, TLR9, DNAH1, BAP1, PHF7, NISCH, STAB1, ITIH3, ITIH4, ANK2, CAMK2D, ELOVL7, ZNF184, CTSB, SORBS3, PDLIM2, C8orf58, BIN3, SH3GL2, FAM171A1, GALC, COQ7, TOX3, ATP6V0A1, PSMC3I, TUBG2, GBA-SYT11, RAB7L1-NUCKS1, SIPA1L2, ACMSD-TMEM163, STK39, KRT8P25-APOOP2, NMD3, TMEM175-GAK-DGKQ, BST1, HLA-DQB1, GPNMB, FGF20, MMP16, ITGA8, INPP5F, M1R4697, LRRK2, CCDC62, GCH1, TMEM229B, VPS13C, BCKDK-STXIB, SREBF1-RAI1, MAPT, SPPL2B, DDRGKI, USP25, FCGR2A, VAMP4, KCNS3, KCNIP3, L1NC00693, KPNA1, MED12L, SPTSSB, LCORL, CLCN3, PAM, C5orf24, TRIM40, RIMS1, RPS12, GS1-124K5.11, FAM49B, UBAP2, GBF1, RNF141, SCAF11, FBRSLI, CAB39L, MBNL2, MIPOLI, RPS6KL1, CD19, NOD2, CNOT1, CHRNBI, UBTF, FAM171A2, BRIP1, DNAH17, ASXL3, MEX3C, CRLS1, DYRKIA and combinations thereof, wherein the genetic variant is a SNP selected from the group consisting of rs34043159, GSA-rs353116, rs4073221, rs12497850, rs143918452, rs78738012, rs2694528, rs9468199, rs2740594, rs2280104, rs13294100, rs10906923, rs8005172, rs11343, rs4784227, rs601999, rs35749011, rs10797576, rs6430538, rs1474055, rs115185635, rs34016896, rs34311866, rs11724635, rs9275326, rs199347, rs591323, rs60298754, rs7077361, rs117896735, rs329648, rs11060180, rs11158026, rs1555399, rs2414739, rs14235, rs11868035, rs17649553, rs113579895, rs62120679, rs8118008, rs2823357, rs6658353, rs11578699, rs76116224, rs2042477, rs6808178, rs55961674, rs11707416, rs1450522, rs34025766, rs62333164, rs26431, rs11950533, rs9261484, rs12528068, rs75859381, rs76949143, rs2086641, rs6476434, rs10748818, rs7938782, rs7134559, GSA-rs11610045, rs9568188, rs4771268, rs12147950, rs3742785, rs2904880, rs6500328, rs200564078, rs12600861, rs2269906, rs850738, rs61169879, rs666463, rs1941685, rs8087969, rs77351827, rs2248244, rs4613239, rs1474055 and combinations thereof.

In one example, the genetic variants are rs6826785, rs141336855, rs6679073, rs2292056, rs16846351, rs3816248, rs12278023, rs9638616, rs1887316, rs246814, rs4130047, rs11724635, rs34311866, rs1941685, rs2414739, rs591323, rs75859381, rs9468199, rs13294100, rs11060180, rs34025766, rs12528068, rs6476434, rs7938782, rs7134559, GSA-rs11610045, rs3742785, rs2269906 and rs1474055.

In another example, the genetic variants are rs6826785, rs141336855, rs6679073, rs2292056, rs16846351, rs3816248, rs12278023, rs9638616, rs1887316, rs31244, rs4130047, rs11724635, rs34311866, rs1941685, rs2414739, rs591323, rs75859381, rs9468199, rs13294100, rs11060180, rs34025766, rs12528068, rs6476434, rs7938782, rs7134559, GSA-rs11610045, rs3742785, rs2269906 and rs1474055.

In another example, the genetic variants are rs6826785, rs141336855, rs6679073, rs2292056, rs16846351, rs3816248, rs12278023, rs9638616, rs1887316, rs246814, rs4130047, rs34043159, GSA-r5353116, rs4073221, rs12497850, rs143918452, rs78738012, rs2694528, rs9468199, rs2740594, rs2280104, rs13294100, rs10906923, rs8005172, rs11343, rs4784227, rs601999, rs35749011, rs10797576, rs6430538, rs1474055, rs115185635, rs34016896, rs34311866, rs11724635, rs9275326, rs199347, rs591323, rs60298754, rs7077361, rs117896735, rs329648, rs11060180, rs11158026, rs1555399, rs2414739, rs14235, rs11868035, rs17649553, rs113579895, rs62120679, rs8118008, rs2823357, rs6658353, rs11578699, rs76116224, rs2042477, rs6808178, rs55961674, rs11707416, rs1450522, rs34025766, rs62333164, rs26431, rs11950533, rs9261484, rs12528068, rs75859381, rs76949143, rs2086641, rs6476434, rs10748818, rs7938782, rs7134559, GSA-rs11610045, rs9568188, rs4771268, rs12147950, rs3742785, rs2904880, rs6500328, rs200564078, rs12600861, rs2269906, rs850738, rs61169879, rs666463, rs1941685, rs8087969, rs77351827, rs2248244, rs4613239 and rs1474055.

In yet another example, the genetic variants are rs6826785, rs141336855, rs6679073, rs2292056, rs16846351, rs3816248, rs12278023, rs9638616, rs1887316, rs31244, rs4130047, rs34043159, GSA-r5353116, rs4073221, rs12497850, rs143918452, rs78738012, rs2694528, rs9468199, rs2740594, rs2280104, rs13294100, rs10906923, rs8005172, rs11343, rs4784227, rs601999, rs35749011, rs10797576, rs6430538, rs1474055, rs115185635, rs34016896, rs34311866, rs11724635, rs9275326, rs199347, rs591323, rs60298754, rs7077361, rs117896735, rs329648, rs11060180, rs11158026, rs1555399, rs2414739, rs14235, rs11868035, rs17649553, rs113579895, rs62120679, rs8118008, rs2823357, rs6658353, rs11578699, rs76116224, rs2042477, rs6808178, rs55961674, rs11707416, rs1450522, rs34025766, rs62333164, rs26431, rs11950533, rs9261484, rs12528068, rs75859381, rs76949143, rs2086641, rs6476434, rs10748818, rs7938782, rs7134559, GSA-rs11610045, rs9568188, rs4771268, rs12147950, rs3742785, rs2904880, rs6500328, rs200564078, rs12600861, rs2269906, rs850738, rs61169879, rs666463, rs1941685, rs8087969, rs77351827, rs2248244, rs4613239 and rs1474055.

It is well known in epidemiology that ethnic variations exist and contribute to the prevalence and etiology of various diseases. In PD, it is known that different ethnic populations have different rates of occurrence, for example, Caucasians vs. Asians. It is also known that different ethnic populations have different disease progression, such as in the development of motor symptoms.

It is understood, with the underlying distinct genetic risk factors and etiologies, that patients with the same disease may show different results to the same method of diagnosis. They may also respond differently to the same treatment. There may be ethnic differences in allele frequencies and effect sizes. For example, a SNP of a gene may be strongly associated with the Asian population, but not European population, suggesting potential genetic or allelic heterogeneity at this gene. A previously identified genetic variant may be limited in use by allelic heterogeneity in a different population. Therefore, the methods of the invention may also be applied to various ethnic populations.

In one example, the methods of the present invention may be used in a subject of Asian ethnicity or ancestry. In another example, the subject is of Han Chinese ancestry or Chinese ethnicity or ancestry with no mixed ancestry, or a South Korean ethnicity or ancestry. In the present disclosure, the terms “ancestry” and “ethnicity” are of the same meaning and hence can be used interchangeably.

In one example, the ancestry or ethnicity of the subject is determined by PCA.

PCA may be used to measure the genetic distance and relatedness between an individual and one or more other individuals of known ancestry or ethnicity. Comparison of the genetic distance between the individual with other individuals of known ancestry or ethnicity allows the ancestry or ethnicity of the individual to be mapped or determined. For example, PCA can be used to confirm the ancestry or ethnicity of an individual as samples of a specific ancestry or ethnicity are expected to cluster together. In another example, PCA can be used to disprove the ancestry or ethnicity of an individual or identify an individual with mixed ancestry when a sample obtained from the individual does not cluster with samples of known ancestry or ethnicity.

In one example, PCA may be used to determine an individual as being of Asian ethnicity or ancestry. In another example, PCA may be used to determine an individual as being of Han Chinese ancestry or Chinese ethnicity or ancestry with no mixed ancestry. In yet another example, PCA may be used to determine an individual as being of South Korean ethnicity or ancestry.

In another aspect, the present invention refers to a kit comprising one or more reagents to detect the presence of a genetic variant at the loci of one or more genes selected from the group consisting of SV2C, WBSCR17, PARK16, ITPKB, MCCC1, SNCA, FAM47E-SCARB2, FYN, DLG2, LRRK2, RIT2 and combinations thereof in a sample, together with instructions for use.

In one example, the kit comprises one or more reagents to detect the presence of a genetic variant at the loci of SV2C and WBSCR17 genes.

In another example, the kit comprises one or more reagents to detect the presence of a genetic variant at the loci of SV2C, WBSCR17, PARK16, ITPKB, MCCC1, SNCA, FAM47E-SCARB2, FYN, DLG2, LRRK2 and RIT2.

In one example, in addition to the 11 genes listed in the foregoing, the kit may further comprise reagents to detect the presence of a genetic variant at the loci of one or more genes selected from the group consisting of ILIR2, SCN3A, SATB1, NCKIPSD, CDC71, ALAS1, TLR9, DNAH1, BAP1, PHF7, NISCH, STAB1, ITIH3, ITIH4, ANK2, CAMK2D, ELOVL7, ZNF184, CTSB, SORBS3, PDLIM2, C8orf58, BIN3, SH3GL2, FAM171A1, GALC, COQ7, TOX3, ATP6V0A1, PSMC3I, TUBG2, GBA-SYT11, RAB7L1-NUCKS1, SIPA1L2, ACMSD-TMEM163, STK39, KRT8P25-APOOP2, NMD3, TMEM175-GAK-DGKQ, BSTI, HLA-DQB1, GPNMB, FGF20, MMP16, ITGA8, INPP5F, MIR4697, LRRK2, CCDC62, GCH1, TMEM229B, VPS13C, BCKDK-STXIB, SREBF1-RAI1, MAPT, SPPL2B, DDRGKI, USP25, FCGR2A, VAMP4, KCNS3, KCNIP3, LINC00693, KPNA1, MED12L, SPTSSB, LCORL, CLCN3, PAM, C5orf24, TRIM40, RIMS1, RPS12, GS1-124K5.11, FAM49B, UBAP2, GBF1, RNF141, SCAF11, FBRSLI, CAB39L, MBNL2, MIPOL1, RPS6KL1, CD19, NOD2, CNOT1, CHRNB1, UBTF, FAM171A2, BRIP1, DNAH17, ASXL3, MEX3C, CRLS1, DYRKIA and combinations thereof.

In one example, in addition to detecting a genetic variant at the loci of the SV2C, WBSCR17, PARK16, ITPKB, MCCC1, SNCA, FAM47E-SCARB2, FYN, DLG2, LRRK2 and RIT2, the kit further comprises one or more reagents to detect the presence of a genetic variant at the loci of BSTI, GAK, ASXL3, VPS13C, FGF20, RPS12, ZNF184, SH3GL2, CCDC62, LCORL, RIMS1, UBAP2, RNF141, SCAF11, FBRSLI, RPS6KL1, UBTF and STK39 genes.

In another example, in addition to detecting a genetic variant at the loci of the SV2C, WBSCR17, PARK16, ITPKB, MCCC1, SNCA, FAM47E-SCARB2, FYN, DLG2, LRRK2 and RIT2, the kit further comprises one or more reagents to detect the presence of a genetic variant at the loci of the ILIR2, SCN3A, SATB1, NCKIPSD, CDC71, ALAS1, TLR9, DNAH1, BAP1, PHF7, NISCH, STAB1, ITIH3, ITIH4, ANK2, CAMK2D, ELOVL7, ZNF184, CTSB, SORBS3, PDLIM2, C8orf58, BIN3, SH3GL2, FAM171A1, GALC, COQ7, TOX3, ATP6V0A1, PSMC3I, TUBG2, GBA-SYT11, RAB7L1-NUCKS1, SIPA1L2, ACMSD-TMEM163, STK39, KRT8P25-APOOP2, NMD3, TMEM175-GAK-DGKQ, BST1, HLA-DQB1, GPNMB, FGF20, MMP16, ITGA8, INPP5F, M1R4697, LRRK2, CCDC62, GCH1, TMEM229B, VPS13C, BCKDK-STXIB, SREBF1-RAI1, MAPT, SPPL2B, DDRGKI, USP25, FCGR2A, VAMP4, KCNS3, KCNIP3, L1NC00693, KPNA1, MED12L, SPTSSB, LCORL, CLCN3, PAM, C5orf24, TRIM40, RIMS1, RPS12, GS1-124K5.11, FAM49B, UBAP2, GBF1, RNF141, SCAF11, FBRSLI, CAB39L, MBNL2, MIPOLI, RPS6KL1, CD19, NOD2, CNOT1, CHRNBI, UBTF, FAM171A2, BRIP1, DNAH17, ASXL3, MEX3C, CRLS1 and DYRKIA genes.

In one example, in the kit, the one or more reagents comprises a reagent to isolate a nucleic acid from the sample and at least one primer for amplification of a sequence encoding the genetic variant or part thereof. In another example, the one or more reagents comprises a reagent to isolate a nucleic acid from the sample and at least one probe for amplification of a sequence encoding the genetic variant or part thereof. In yet another example, the one or more reagents comprises a reagent to isolate a nucleic acid from the sample and at least one primer and at least one probe for amplification of a sequence encoding the genetic variant or part thereof.

In one example, the kit of the present invention may be used to identify whether a subject is at risk of developing PD, to identify whether a subject is suffering from PD or whether a subject is in need of early therapeutic intervention for PD.

In another example, kit of the present invention may be used to determine the prognosis of a subject with PD or a subject at risk of developing PD.

In yet another example, the kit of the present invention may be used to calculate a PRS of a subject of developing PD.

It will be understood that the kit of the present invention may be used for one or more of the uses recited herein.

The term “sequence encoding the genetic variant” may refer to any portion of the chromosome that encodes the genetic variant or SNP, including coding and non-coding regions. Coding regions may refer exon. Non-coding regions may refer to regulatory regions or regions without known regulatory functions. Examples of non-coding regions include, but are not limited to, intron, 5′ UTR, 3′UTR, and regulatory regions such as enhancer, transcription factor binding domain and DNA methylation region. In other words, the term “sequence encoding the genetic variant” may refer to the sequence encoding the gene or the sequence affecting the gene or the disease. In some examples, it may refer to the sequence encoding the isoforms of the gene. In one example, it refers to exon. In another example, it refers to intron. In another example, it refers to the promoter region. In another example, it refers to the enhancer region. In yet another example, it refers to the transcription factor binding region.

It will be well understood to one of skill in the art that genetic variant may be detected by a variety of genotyping methods. Examples of methods to detect genetic variation include but are not limited to polymerase chain reaction (PCR), quantitative PCR (qPCR), microarray, real time-PCR (RT-PCR) and Northern blot. Other examples of detection methods include but are not limited to restriction fragment length polymorphism identification (RFLPI) of genomic DNA, random amplified polymorphic detection (RAPD) of genomic DNA, amplified fragment length polymorphism detection (AFLPD), polymerase chain reaction (PCR), DNA sequencing, allele specific oligonucleotide (ASO) probes, and hybridization to DNA microarrays or beads, (epi)GBS (Genotyping by sequencing), RADseq. In some examples, the detection method may be NGS or massive parallel DNA sequencing. In one example, the detection method may be microarray.

It will also be understood to one of skill in the art that a variety of detection reagents may be used to detect the genetic variation. Examples of detection reagents include but are not limited to primers, probes and complementary nucleic acid sequences that hybridize to the gene.

In another example, in the method or the kit as described in the foregoing, the sample is selected from the group consisting of an oral tissue sample, scraping, or wash or a biological fluid sample, saliva, urine or blood or post mortem brain tissue. Examples of the sample includes but is not limited to blood, serum, saliva, urine, cerebrospinal fluid or bone marrow fluid. In one example, the sample is blood. Some other examples of the sample includes but is not limited to fresh tissue, frozen fresh tissue, paraffin embedded tissue or formalin fixed paraffin embedded tissue. In another example, the samples refers to DNA, RNA or protein extracted from one of various types of tissue. In another example, the sample is DNA extracted from one of various types of tissues. In another example, the sample is DNA extracted from blood collected from subjects.

The present invention also refers to a PD biomarker. A PD biomarker may be a combination of genetic variants at the loci of one or more genes.

In one aspect, the present invention refers to a PD biomarker, wherein the biomarker is a genetic variant at the loci of one or more genes selected from the group consisting of SV2C, WBSCR17, PARK16, ITPKB, MCCC1, SNCA, FAM47E-SCARB2, FYN, DLG2, LRRK2, RIT2i and combinations thereof.

In one example, the biomarker is a genetic variant at the loci of SV2C and WBSCR17 genes.

In another example, the biomarker is a genetic variant at the loci of SV2C, WBSCR17, PARK16, ITPKB, MCCC1, SNCA, FAM47E-SCARB2, FYN, DLG2, LRRK2 and RIT2.

The biomarker can be a genetic variant of different types, for example, SNV or SNP. In one example, the biomarker is a SNP at the loci of SV2C and WBSCR17.

In another example, the biomarker is a SNP at the loci of SV2C, WBSCR17, PARK16, ITPKB, MCCC1, SNCA, FAM47E-SCARB2, FYN, DLG2, LRRK2 and RIT2.

In one example, the biomarker is a SNP selected from the group consisting of rs9638616, rs246814, rs31244 and combinations thereof.

In another example, the biomarker is a SNP selected from the group consisting of rs6826785, rs141336855, rs6679073, rs2292056, rs16846351, rs3816248, rs12278023, rs9638616, rs1887316, rs246814, rs31244, rs4130047 and combinations thereof.

In another example, the biomarker is an effect allele or risk allele of the genetic variant, wherein the effect allele or risk allele of rs6826785 is cytosine (C), the effect allele of rs141336855 is thymine (T), the effect allele of rs6679073 is adenine (A), the effect allele of rs2292056 is guanine (G), the effect allele of rs16846351 is guanine (G), the effect allele of rs3816248 is cytosine (C), the effect allele of rs12278023 is cytosine (C), the effect allele of rs9638616 is thymine (T), the effect allele of rs1887316 is adenine (A), the effect allele of rs246814 is thymine (T), the effect allele of rs31244 is guanine (G), and the effect allele of rs4130047 is cytosine (C).

The biomarker can be used to, but not limited to, 1) identify whether a subject is at risk of developing PD, whether a subject is suffering from PD, or whether a subject is in need of early therapeutic intervention for PD; 2) determine the prognosis of a subject with PD or a subject at risk of developing PD including identification of therapeutic needs; 3) calculate a PRS of a subject of developing PD; or 4) stratify subjects who are suffering from PD or at risk of developing PD. It will be understood that the biomarker of the present invention may be used for one or more of the uses recited herein.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non- limiting examples. In addition, where features or aspects of the invention are described in terms of Markush groups, those skilled in the art will recognize that the invention is also thereby described in terms of any individual member or subgroup of members of the Markush group.

### Experimental Section

Non-limiting examples of the invention and comparative examples will be further described in greater detail by reference to specific Examples, which should not be construed as in any way limiting the scope of the invention.

Methods

Patient Recruitment and Sample Collection

Patients and ethnically- and regionally-matched controls were recruited by thirteen independent centres and study groups from six regions across East Asia. A total of 35,994 subjects were recruited, out of which 34,162 DNA samples (94.9% of recruited subjects) passed quality control for genotyping and 31,575 (92.4% of genotyped samples) were included in the final analysis. Patients were diagnosed with PD using the United Kingdom Parkinson's Society Brain Bank Criteria. The subjects' consent was obtained according to the Declaration of Helsinki. Blood samples were collected from each participant and DNA extraction was performed. This study was approved by the ethics committees or institutional review boards of the respective institutions (SingHealth Centralized Institutional Review Board CIRB 2002/008/A and 2019/2334 and Nanyang Technological University Institutional Review Board IRB-2016-08-011).

GWAS Genotyping and Statistical Analysis

Samples (N=34,162) were genotyped on the Illumina Infinium Global Screening Array −24 v2.0 for 759,993 SNPs. Samples were grouped into five regions: Singapore/Malaysia, Hong Kong, Taiwan, mainland China and South Korea. Genotype data from each batch was exported and converted to forward strand. Samples with extreme sample heterozygosity, gender inconsistencies, call rates <95%, SNPs with call rates <95%, minor allele frequencies (MAF) <1% and Hardy-Weinberg equilibrium (HWE) P<10⁻³ in controls and/or P<10⁻⁶ in all samples as well as all non-autosomal SNPs (X, Y and mitochondrial chromosomes) were excluded.

After performing identity-by-descent analysis using overlapping genotyped SNPs in PLINK and first-degree relative pair identification; the relative with a lower sample call rate was excluded. Principal components analysis was also run on 82,324 independent genotyped SNPs (pruned with pairwise r²<0.1 in a window of 500 SNPs, sliding in steps of 50) after exclusion of SNPs in the five conserved long-range linkage disequilibrium (LD) regions in Chinese. Outliers on the first six principal components were then excluded and principal components analysis was re-run in the remaining samples. 31,575 samples remained for the final analysis.

The software IMPUTE version 2 was used for imputation of untyped SNPs in each dataset following pre-phasing using SHAPEIT2, and using the multi-ethnic 1000 genomes Phase 3 reference panel consisting of 77,818,332 biallelic SNP genotypes in 2,504 individuals from Africa, East and South Asia, Europe, and the Americas. The imputation was ran separately for each of the five regions. Further stringent quality control filtering was run at the SNP level, excluding those with MAF <1%, info score <0.8, HWE Pin controls <10⁻³, HWE Pin all samples<10⁻⁶. All the 11 genome-wide significant SNPs were confirmed to have either good genotyping clusters or high imputation info scores.

Logistic regression analyses was run on genotype dosages adjusting for the first three principal components using SNPTEST. The results were combined using a fixed-effects inverse variance meta-analysis in PLINK.

Polygenic Risk Calculations

PRS were calculated in 2,536 PD cases and 21,840 population- based controls from Singapore and Malaysia. Weighted PRS were calculated based on sum of high-risk alleles weighted by their effect sizes (beta) that were calculated based on meta-analysis across five Asian datasets (11 Asian SNPs) or reported in the respective publications (Chang et al, 2017; Nalls et al, 2014; Nalls et al, 2019) (78 European SNPs). For polygenic risk scores combining Asian and European SNPs, 80 SNPs were included, whereby only the Asian SNP was considered at each of the nine loci that overlapped between the Asian and European PRS model. PRS cut-offs for the top and bottom 5% and 10% were determined based on the 21,840 population controls, and numbers of PD cases within each score range were then determined to estimate fold-difference in risk between the two extreme groups.

Fraction of Variance and Area Under Curve Analysis

The percentage of the total variance explained was estimated by calculating Nagelkerke's pseudo R² using the fmsb package, entering SNP genotypes and affection status into the glm function in R (v 3.5.0). Receiver-operating characteristic (ROC) curves and area under the curve (AUC) estimates were done using the pROC package, using the bootstrap test (n=100) to assess differences between two ROC curves.

Replication in European-Ancestry and Japanese Samples

SNPs within the two novel loci were analyzed in 988 PD cases and 2,521 controls from Japan and SNPs in high LD (r²>0.9) were identified using SNiPA. The top SNPs in the largest and most recent European-ancestry PD GWAS (56,306 cases, 1,417,791 controls recruited from North America, Europe, Asia and Australia) from the IPDGC were analyzed.

Results

### EXAMPLE 1

### Meta-GWAS of PD Cases and Controls from Five Regions

A total of 31,575 samples remained after quality control filtering, consisting of 6,724 PD cases 24,851 controls from China (2,279 cases, 2,021 controls), Taiwan (216 cases, 225 controls), Hong Kong (199 cases, 166 controls), South Korea (1,494 cases, 599 controls) and Chinese participants from Singapore and Malaysia (2,536 cases, 21,840 controls). Association statistics were combined using fixed effects meta-analysis at a total of 5,843,213 SNPs (MAF≥1%; λ_{GC}=1.082; λ₁₀₀₀=1.0077; λ_{GC} for MAF≥5%=1.092; λ₁₀₀₀=1.0087; LD score intercept=1.02) that were genotyped or successfully imputed at high quality across all five datasets. Sensitivity analyses using leave-one-out meta-analyses suggested that the effect size estimates were not driven by any single study (Table 2).

Table 2 Sensitivity analyses using leave-one-out meta-analysis using correlation between beta estimated across all 5,843,213 SNPs using all 5 datasets and beta estimated when one dataset is left out. For the 11 genome-wide significant loci, beta values from each meta-analysis (fixed effects) are shown for the lead SNP.

TABLE 2
Sensitivity analyses using leave-one-out meta-analysis using correlation between
beta estimated across all 5,843, 213 SNPs using all 5 datasets and beta estimated
when one dataset is left out. For the 11 genome-wide significant loci, beta
values from each meta-analysis (fixed effects) are shown for the lead SNP.

| | | All 5 | Exclude | Exclude | Exclude | Exclude | Exclude |
|---|---|---|---|---|---|---|---|
| Lotus | SNP | (Beta) | China | Singapore/Malaysia | Korea | Taiwan | Hongkong |
| Correlation | — | — | 0.86 | 0.66 | 0.95 | 0.99 | 0.99 |
| PARK16 | rs6679073 | 0.21 | 0.18 | 0.30 | 0.20 | 0.21 | 0.21 |
| ITPKB | rs16846351 | 0.29 | 0.26 | 0.22 | 0.34 | 0.29 | 0.28 |
| MCCC1 | rs2292056 | −0.19 | −0.19 | −0.18 | −0.19 | −0.20 | −0.19 |
| FAM47E-SCARB2 | rs3816248 | −0.14 | −0.12 | −0.20 | −0.12 | −0.14 | −0.13 |
| SNCA | rs6826785 | 0.29 | 0.30 | 0.29 | 0.28 | 0.30 | 0.30 |
| SV2C | rs246814 | 0.22 | 0.25 | 0.17 | 0.22 | 0.22 | 0.22 |
| FYN | rs1887316: | −0.19 | −0.23 | −0.15 | −0.18 | −0.19 | −0.19 |
| WBSCR17 | rs9638616 | 0.13 | 0.15 | 0.13 | 0.12 | 0.13 | 0.12 |
| DLG2 | rs12278023 | −0.13 | −0.11 | −0.16 | −0.12 | −0.13 | −0.13 |
| LRRK2 | rs141336855 | 0.69 | 0.64 | 0.72 | 0.69 | 0.70 | 0.69 |
| RIT2 | rs4130047 | 0.13 | 0.13 | 0.09 | 0.14 | 0.13 | 0.13 |

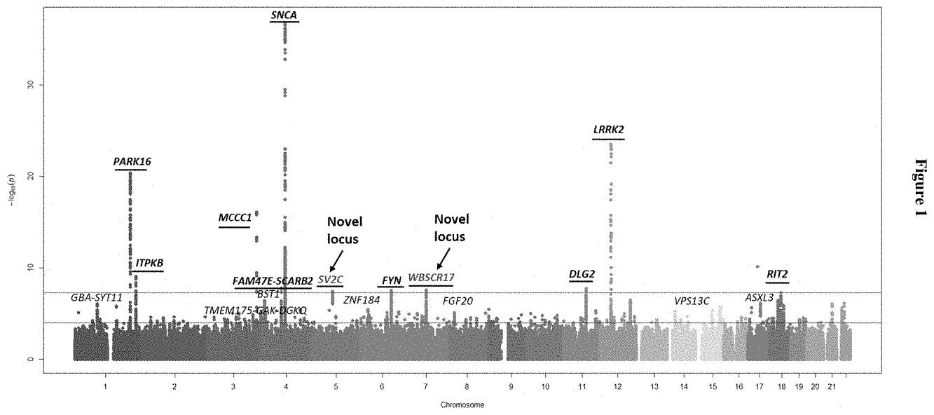

This meta-analysis revealed eleven genome-wide significant loci out of which nine were previously described (PARK16, ITPKB, MCCC1, SNCA, FAM47E-SCARB2, DLG2, LRRK2, RIT2 and FYN) (FIG. 1). Two new associations were identified at SV2C and WBSCR17. Strong association (P<1×10⁻⁵) was also observed at seven other loci that have previously (GBA-SYT11, BST1, TMEM175-GAK-DGKQ, ZNF184, FGF20, VPS13C, ASXL3) been reported to be associated with PD in Europeans (FIG. 1). Out of the sixteen previously-reported loci with P<1×10⁻⁵, the top-associated SNP was highly correlated (r²>0.75) to the reported European SNP within seven loci. Allelic heterogeneity was observed at LRRK2, ITPKB, ZNF184, FAM47E-SCARB2 and GBA/SYT11 in which the top Asian SNP was independent of the reported European SNP, and LD differences at SNCA, FYN, VPS13C and ASXL3 (Table 3), thus demonstrating differences in the underlying genetic architecture between Asians and Europeans at overlapping loci.

### EXAMPLE 2

### Two Novel Genome-Wide Significant Loci

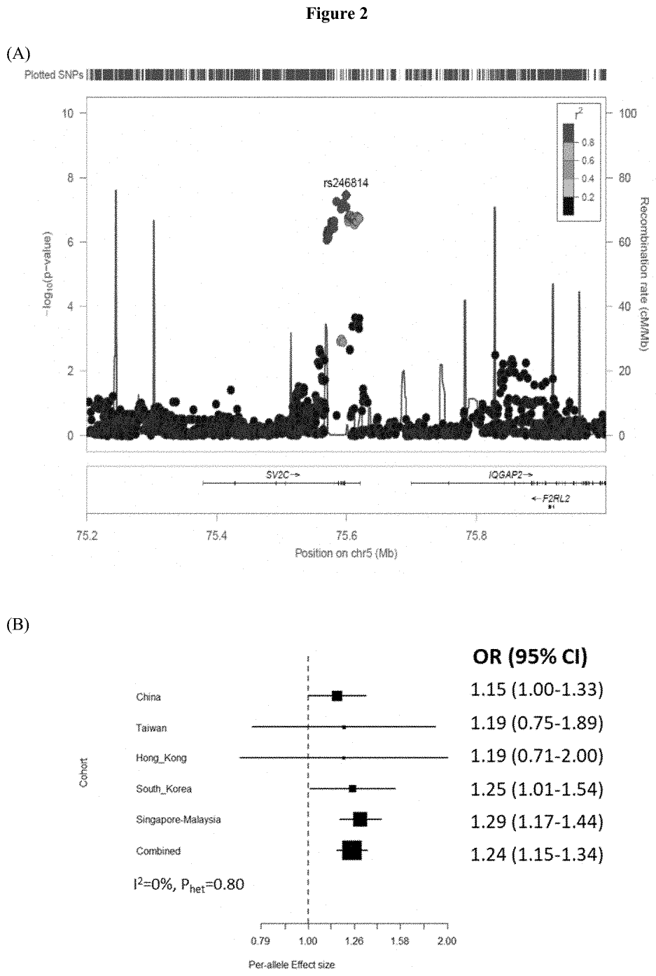

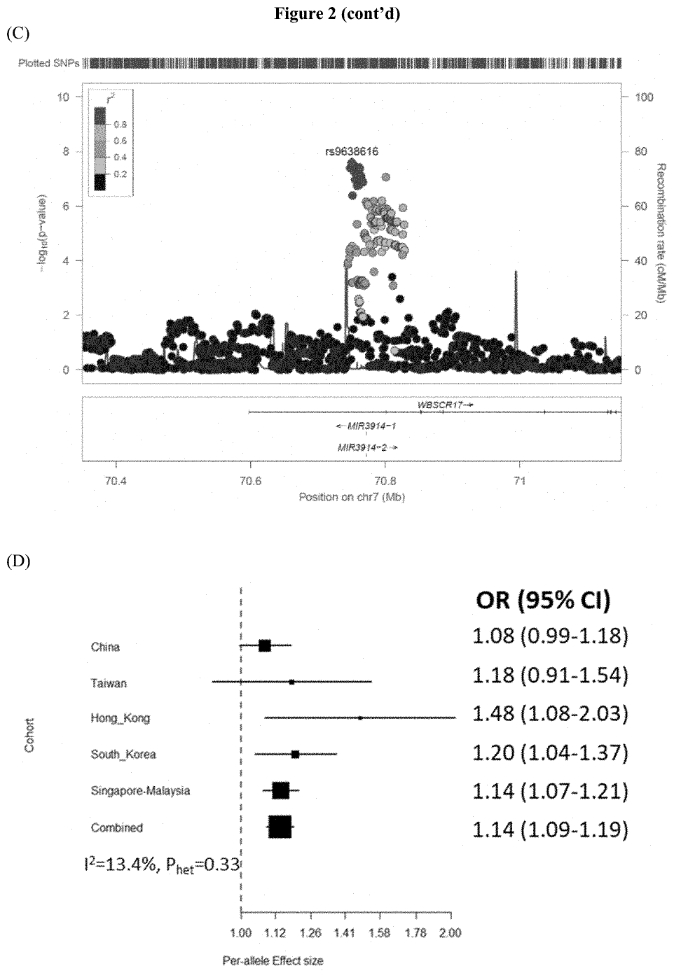

Genome-wide significant association was observed at rs246814 (OR=1.24, 95% Cl=1.15−1.34, P=3.48×10⁻⁸) located within an intron of the SV2C gene (FIG. 2A, Table 4). Consistent association was observed across all five East Asian datasets (I²=0, Pₕₑₜ=0.79). This SNP is in complete LD (r²=1 in 1000 Genomes data and >0.96 in the present samples) with a missense variant p.Asp543Asn (rs31244) within SV2C (OR=1.24, 95% CI=1.14-1.33, P=6.22×10⁻⁸). Although this nonsynonymous change is predicted by SIFT and PolyPhen to be tolerated and benign respectively, it occurs within an extracellular/luminal domain of SV2C and may affect N-linked glycosylation of this domain via the creation of a new glycosylation site (Asn543-Asp544-Thr545). It also tags SNPs located within potential transcription factor binding motifs and DNase hypersensitivity sites. SV2C is expressed in the basal ganglia and dopaminergic neurons, and has previously been evaluated as a functional PD candidate gene because of its restricted expression in brain region relevant to PD.

Genome-wide significant association was also observed at a second novel locus tagged by rs9638616 (OR=1.14, 95% CI=1.09-1.19, P=2.53×10⁻⁸) (FIG. 2B, Table 4). This SNP is located within an intron of the WBSCR17 gene and near genes encoding microRNAs mir-3914-1 and mir3914-2. Similarly, consistent association was observed across the five datasets (I²=13.4%, Pₕₑₜ=0.32). Neither of these two genes has previously been implicated in PD.

TABLE 3
Allele frequency and pairwise linkage disequilibrium between top-associated SNPs
identified in this study vs. reported SNPs at overlapping loci with P < 10⁻⁵.

| | | | | | | gnomAD | | | |
|---|---|---|---|---|---|---|---|---|---|
| | | | | | gnomAD | allele | 1000 | 1000 | |
| | | | | Ref/Alt | allele | frequency | genomes | genomes | |
| | | | | Allele | frequency | Europeans | r2/D′ in | r2/D′ in | Independent |
| Locus | SNP type | SNP | P | (b37) | East Asians | (non-Finnish) | Asians | Europeans | signals? |
| SNCA | Top | rs6826785 | 1.86E−37 | T/C | 53.8% | 6.2% | 0.479/−0.836 | 0/0 | LD |
| | Reported | rs356182 | 7.15E−20 | G/A | 33.2% | 65.1% | | | differences |
| LRKK2 | Top | rs141336855 | 2.97E−24 | G/T | 2.8% | 0.0% | 0/0 | 0/0 | Yes |
| | Reported | rs76904798 | 0.707 | C/T | 4.4% | 12.8% | | | |
| PARK16 | Top | rs6679073 | 4.10E−21 | C/A | 53.7% | 27.5% | 0.942/0.996 | 0/0 | No |
| | Reported | rs823118 | 9.76E−18 | C/T | 54.5% | 55.9% | | | |
| MCCC1 | Top | rs2292056 | 8.14E−17 | T/G | 58.0% | 19.3% | 0.968/0.996 | 1/1 | No |
| | Reported | rs12637471 | 1.49E−16 | G/A | 57.5% | 19.3% | | | |
| ITPKB | Top | rs16846351 | 8.16E−10 | C/G | 6.4% | 0.0% | 0/0 | 0/0 | Yes |
| | Reported | rs4653767 | 3.18E−07 | T/C | 28.9% | 27.0% | | | |
| FAM47E- | Top | rs3816248 | 1.53E−08 | T/C | 36.7% | 16.1% | 0/0 | 0/0 | Yes |
| SCARB2 | Reported | rs6812193 | 0.1825 | C/T | 8.0% | 36.1% | | | |
| DLG2 | Top | rs12278023 | 1.83E−08 | T/C | 49.2% | 43.9% | 0.765/0.973 | 0.906/0.959 | No |
| | Reported | rs3793947 | 3.26E−07 | G/A | 47.0% | 44.5% | | | |
| FYN | Top | rs1887316 | 2.89E−08 | G/A | 11.8% | 7.6% | 0.217/0.948 | 0.348/0.859 | LD |
| | Reported | rs997368 | 5.57E−06 | A/G | 34.1% | 17.6% | | | differences |
| RIT2 | Top | rs4130047 | 5.04E−08 | T/C | 37.9% | 33.0% | 0.983/1 | 1/1 | No |
| | Reported | rs12456492 | 1.32E−07 | A/G | 38.0% | 33.0% | | | |
| GBA-SYT11 | Top | rs146532106 | 1.96E−06 | G/C | 4.5% | 0.0% | 0/0 | 0/0 | Yes |
| | Reported | rs35749011 | too rare | G/A | 0.0% | 1.4% | | | |
| BST1 | Top | rs6449168 | 3.77E−07 | C/T | 63.3% | 23.1% | 0.930/−0.979 | 0.401/−0.993 | No |
| | Reported | rs11724635 | 1.53E−06 | C/A | 35.8% | 54.6% | | | |
| TMEM175- | Top | rs34311866 | 7.03E−06 | T/C | 12.9% | 19.0% | 1/1 | 1/1 | No |
| GAK-DGKQ | Reported | rs34311866 | 7.03E−06 | T/C | 12.9% | 19.0% | | | |
| FGF20 | Top | rs532233 | 7.82E−06 | T/C | 44.5% | 55.9% | 0.791/0.948 | 0.330/0.956 | No |
| | Reported | rs591323 | 7.59E−05 | G/A | 44.0% | 29.2% | | | |
| VPS13C | Top | rs56287080 | 4.93E−06 | G/A | 7.7% | 13.7% | 0.352/−0.970 | 0.158/−0.560 | LD |
| | Reported | rs2414739 | 4.49E−05 | G/A | 83.5% | 71.1% | | | differences |
| ASXL3 | Top | rs7228309 | 3.74E−07 | C/T | 54.6% | 39.9% | 0.158/0.955 | 0.508/0.940 | LD |
| | Reported | rs1941685 | 5.90E−05 | G/T | 88.9% | 50.1% | | | differences |
| ZXF134 | Top | rs9379967 | 3.56E−06 | T/C | 12.9% | 10.6% | 0/0 | 0/0 | Yes |
| | Reported | rs9468199 | 7.20E−03 | G/A | 20.9% | 16.8% | | | |

TABLE 4
Association and meta-analysis results at SV2C and WBSCR17.

| | | | | | | | |
|---|---|---|---|---|---|---|---|
| | MAF | MAF | | | | | |
| Study | cases | controls | OR | 95% CI | P | I² | Pₕₑₜ |

SV2C rs246814: C

| China | 10.32% | 9.13% | 1.154 | 0.998-1.334 | 0.054 | | |
|---|---|---|---|---|---|---|---|
| Taiwan | 10.51% | 8.74% | 1.193 | 0.755-1.886 | 0.449 | | |
| Hong Kong | 9.79% | 8.67% | 1.192 | 0.711-1.999 | 0.505 | | |
| Korea | 11.92% | 9.63% | 1.247 | 1.007-1.543 | 0.043 | | |
| Singapore-Malaysia | 10.45% | 8.29% | 1.294 | 1.165-1.438 | 1.44E−06 | | |
| Combined Discovery | | | 1.242 | 1.150-1.341 | 3.48E−08 | 0% | 0.801 |
| Japan* | 11.08% | 10.13% | 1.105 | 0.935-1.307 | 0.242 | | |
| UK Biobank | 7.75% | | 1.090 | 0.943-1.261 | 0.245 | | |
| IPDGC all | 8.23% | | 1.072 | 1.037-1.108 | 3.62E−05 | | |
| IPDGC clinical | 8.42% | | 1.129 | 1.057-1.205 | 2.94E−04 | | |

| | | | | | |
|---|---|---|---|---|---|
| Combined Replication (IPDGC all)# | 1.074 | 1.041-1.109 | 9.74E−06 | 0% | 0.923 |
| Combined Discovery + Replication (all) | 1.110 | 1.065-1.130 | 6.02E−10 | 48% | 0.062 |
| Combined Replication (IPDGC clinical) | 1.120 | 1.059-1.185 | 7.80E−05 | 0% | 0.900 |
| Combined Discovery + Replication (clinical) | 1.161 | 1.109-1.215 | 1.17E−10 | 0% | 0.498 |

WBSCR17 rs9638616: T

| China | 49.24% | 47.22% | 1.081 | 0.992-1.179 | 0.076 | | |
|---|---|---|---|---|---|---|---|
| Taiwan | 48.13% | 44.17% | 1.182 | 0.909-1.536 | 0.213 | | |
| Hong Kong | 47.26% | 38.39% | 1.480 | 1.081-2.026 | 0.014 | | |
| Korea | 56.68% | 52.29% | 1.196 | 1.045-1.369 | 9.50E−03 | | |
| Singapore-Malaysia | 47.06% | 43.27% | 1.139 | 1.073-1.209 | 1.93E−05 | | |
| Combined Discovery | | | 1.137 | 1.086-1.189 | 2.53E−08 | 13.4% | 0.328 |
| Japan* | 41.19% | 40.16% | 1.044 | 0.939-1.160 | 0.428 | | |
| UK Biobank | 31.43% | | 0.973 | 0.894-1.059 | 0.526 | | |
| IPDGC all | 32.44% | | 0.997 | 0.975-1.018 | 0.756 | | |
| IPDGC clinical | 31.81% | | 1.005 | 0.950-1.063 | 0.854 | | |

| | | | | | |
|---|---|---|---|---|---|
| Combined Replication (IPDGC all)# | 0.997 | 0.976-1.018 | 0.765 | 0% | 0.591 |
| Combined Discovery + Replication (all) | 1.020 | 1.001-1.039 | 0.040 | 78.5% | 3.16E−05 |
| Combined Replication (IPDGC clinical) | 1.003 | 0.961-1.047 | 0.888 | 0% | 0.591 |
| Combined Discovery + Replication (clinical) | 1.064 | 1.032-1.098 | 8.37E−05 | 67.1% | 3.40E−03 |

*rs246813 was used as a proxy for rs246814 (r² = 0.99) and rs1317290 was used as a proxy for rs9638616 (r² = 0.90) in data from Japan.
#Replication was performed using either the full IPDGC dataset of 56,306 cases, 1,417,791 controls (all) or the IPDGC clinically-diagnosed subset of 15,056 cases and 12,637 controls (clinical) in which there is no overlap with the UK biobank samples. The Japan and UK Biobank datasets were included in both analyses.

### EXAMPLE 3

### Analysis of European PD Risk SNPs and Loci

The association evidence was evaluated at SNPs and loci previously reported to show genome-wide significant association with PD in European populations (Chang et al, 2017; Nalls et al 2014; Nalls et al, 2019) in the present GWAS meta-analysis results (Table 5, Table 6). Of the 78 SNPs polymorphic in Asian samples, only three showed genome-wide significant association in Asians, and another six were associated at P<1×10⁻⁵ (Table 5). A total of 63 SNPs had OR in same direction (38 with P<0.05), 15 had OR in the opposite direction (all with P>0.05 except MEX3C). It is recognized that the present Asian sample set is smaller than the largest European GWAS and has limited statistical power to validate these loci. However, the fraction of polymorphic SNPs showing same direction of association (63/78=80.8%) and the strong enrichment for significant SNPs (38/78=48.7% at P<0.05; median P=0.055, λ=8.08) suggest a substantial but incomplete overlap in genetic risk between Asian and European populations. At the locus level, SNPs with P<1×10⁻⁵ were observed in 16 of the previously-reported loci (Table 3), while there was no evidence of linked or independent signals crossing P<1×10⁻⁵at the remaining

| | No. of | |
|---|---|---|
| P | variants | Locus names |
| P < 5e−8 | 3 | SNCA, MCCC1, PARK16 |
| P < 1e−5 | 9 | BST1, GAK, ITPKB, RIT2, DLG2, FYN |
| P < 1e−4 | 12 | ASXL3, VPS13C, FGF20 |
| P < 1e−3 | 13 | RPS12 |
| P < 0.01 | 25 | ZNF184, SH3GL2, CCDC62, LCORL, |
| | | RIMS1, UBAP2, RNF141, SCAF11, FBRSL1, |
| | | RPS6KL1, UBTF, STK39 |
| P < 0.05 | 39 | 38 in same direction, 1 in opposite direction |
| | | (MEX3C); see Table 6 |

loci.

Table 5 Variants at reported PD risk loci with P<0.01 in Asian discovery samples. Full SNP rsids and association statistics are listed in see Table 6.

TABLE 6
Lookup of 88 polymorphic SNPs in previously reported
PD loci (Chang, et al, 2017; Nalls et al, 2019)

| Paper | Locus | CHR | BP | SNP |
|---|---|---|---|---|
| Chang et al 2017 | ITPKB | 1 | 226916078 | rs4653767 |
| Chang et al 2017 | IL1R2 | 2 | 102413116 | rs34043159 |
| Chang et al 2017 | SCN3A | 2 | 166133632 | GSA-rs353116 |
| Chang et al 2017 | SATB1 | 3 | 18277488 | rs4073221 |
| Chang eft al 2017 | NCKIPSD, CDC71 | 3 | 48748989 | rs12497850 |
| Chang et al 2017 | ALAS1, TLR9, DNAH1, | 3 | 52816840 | rs143913452 |
| | BAP1, PHF7, NISCH, | | | |
| | STAB1, ITIH3, ITIH4 | | | |
| Chang et al 2017 | ANK2, CAMK2D | 4 | 114360372 | rs78738012 |
| Chang et al 2017 | ELOVL7 | 5 | 60273923 | rs2694528 |
| Chang et al 2017 | ZNF184 | 6 | 27681215 | rs9468199 |
| Chang et al 2017 | CTSB | 8 | 11707174 | rs2740594 |
| Chang et al 2017 | SORBS3, PDLIM2, | 8 | 22525980 | rs2280104 |
| | C8orf38, BIN3 | | | |
| Chang et al 2017 | SH3GL2 | 9 | 17579690 | rs13294100 |
| Chang et al 2017 | FAM171A1 | 10 | 15560598 | rs10906923 |
| Chang et al 2017 | GALC | 14 | 88472612 | rs8005172 |
| Chang et al 2017 | COQ7 | 16 | 19279464 | rs11343 |
| Chang et al 2017 | TOX3 | 16 | 52599188 | rs4784227 |
| Chang et al 2017 | ATP6V0A1, | 17 | 40698158 | rs601999 |
| | PSMC31, TUBG2 | | | |
| Nalls et al 2014 | GBA-SYT11 | 1 | 155135036 | rs35749011 |
| Nalls et al 2014 | RAB7L1-NUCKS1 | 1 | 205723572 | rs823118 |
| Nalls et al 2014 | SIPA1L2 | 1 | 232664611 | rs10797576 |
| Nalls et al 2014 | ACMSD-TMEM163 | 2 | 135539967 | rs6430538 |
| Nalls et al 2014 | STK39 | 2 | 169110394 | rs1474055* |
| Nalls et al 2014 | KRT8P25-APOOP2 | 3 | 87520857 | rs115185635 |
| Nalls et al 2014 | NMD3 | 3 | 160992864 | rs34016896 |
| Nalls et al 2014 | MCCC1 | 3 | 182762437 | rs12637471 |
| Nalls et al 2014 | TMESM175-GAK-DGKQ | 4 | 951947 | rs34311866 |
| Nalls et al 2014 | BST1 | 4 | 15737101 | rs11724635 |
| Nalls et al 2014 | FAM47E-SCARB2 | 4 | 77198986 | rs6812193 |
| Nalls et al 2014 | SNCA | 4 | 90626111 | rs356182 |
| Nalls et al 2014 | HL4-DQBI | 6 | 32666660 | rs9275326 |
| Nalls et al 2014 | GPNMB | 7 | 23293746 | rs199347 |
| Nalls et al 2014 | FGF20 | 8 | 16697091 | rs591323 |
| Nalls et al 2014 | MMP16 | 8 | 89373041 | rs60298754 |
| Nalls et al 2014 | ITGA8 | 10 | 15561543 | rs7077361 |
| Nalls et al 2014 | IXPPSF | 10 | 121536327 | rs117896735 |
| Nalls et al 2014 | DLG2 | 11 | 83544472 | rs3793947 |
| Nalls et al 2014 | MIR4697 | 11 | 133765367 | rs329648 |
| Nalls et al 2014 | LRRK2 | 12 | 40614434 | rs76904798 |
| Nalls et al 2014 | CCDC62 | 12 | 123303586 | rs11060180 |
| Nalls et al 2014 | GCH1 | 14 | 55348869 | rs11158026 |
| Nalls et al 2014 | TMEM229B | 14 | 67984370 | rs1555399 |
| Nalls et al 2014 | VPS13C | 15 | 61994134 | rs2414739 |
| Nalls et al 2014 | BCKDK-STX1B | 16 | 31121793 | rs14235 |
| Nalls et al 2014 | SREBF1-RAI1 | 17 | 17715101 | rs11868035 |
| Nalls et al 2014 | MAPT | 17 | 43994648 | rs17649553/rs113579895 |
| Nalls et at 2014 | RIT2 | 18 | 40673380 | rs12456492 |
| Nalls et al 2014 | SPPL2B | 19 | 2363319 | rs62120679 |
| Nalls et al 2014 | DDRGK1 | 20 | 3168166 | rs8118008 |
| Nalls et al 2014 | USP25 | 21 | 16914905 | rs2823357 |
| Nalls et al 2019 | FCGR2A | 1 | 161469054 | rs6658353 |
| Nalls et al 2019 | VAMP4 | 1 | 171719769 | rs11578699 |
| Nalls et al 2019 | KCNS3 | 2 | 18147848 | rs76116224 |
| Nalls et al 2019 | KCNIP3 | 2 | 96000943 | rs2042477 |
| Nalls et al 2019 | LINC00693 | 3 | 28705690 | rs6808178 |
| Nalls et al 2019 | KPNA1 | 3 | 122196392 | rs55961674 |
| Nalls et al 2019 | MED12L | 3 | 151198965 | rs11707416 |
| Nalls et al 2019 | SPTSSB | 3 | 161077630 | rs1450522 |
| Nalls et al 2019 | LCORL | 4 | 17968811 | rs34025766 |
| Nalls et al 2019 | CLCN3 | 4 | 170583157 | rs62333164 |
| Nalls et al 2019 | PAM | 5 | 102365794 | rs26431 |
| Nalls et al 2019 | C5orf24 | 5 | 134199105 | rs11950533 |
| Nalls et al 2019 | TRIM40 | 6 | 30108683 | rs9261484 |
| Nalls et al 2019 | RIMS1 | 6 | 72487762 | rs12528068 |
| Nalls et al 2019 | FYN | 6 | 112243291 | rs997368 |
| Nalls et al 2019 | RPS12 | 6 | 133210361 | rs75859381 |
| Nalls et al 2019 | GS1-124K5.11 | 7 | 66009851 | rs76949143 |
| Nalls et al 2019 | FAM49B | 8 | 130901909 | rs2086641 |
| Nalls et al 2019 | UBAP2 | 9 | 34946391 | rs6476434 |
| Nalls et al 2019 | GBF1 | 10 | 104015279 | rs10748818 |
| Nalls et al 2019 | RNF141 | 11 | 10558777 | rs7938782 |
| Nalls et al 2019 | SCAF11 | 12 | 46419086 | rs7134559 |
| Nalls et al 2019 | FBRSL1 | 12 | 133063768 | GSA-rs11610045 |
| Nalls et al 2019 | CAB39L | 13 | 49927732 | rs9568188 |
| Nalls et al 2019 | MBNL2 | 13 | 97865021 | rs4771268 |
| Nalls et al 2019 | MIPOL1 | 14 | 37989270 | rs12147950 |
| Nalls et al 2019 | RPS6KL1 | 14 | 75373034 | rs3742785 |
| Nalls et al 2019 | CD19 | 16 | 28944396 | rs2904880 |
| Nalls et al 2019 | NOD2 | 16 | 50736656 | rs6500328 |
| Nalls et al 2019 | CNOT1 | 16 | 58587672 | rs200564078 |
| Nalls et al 2019 | CHRNB1 | 17 | 7355621 | rs12600861 |
| Nalls et al 2019 | UBTF | 17 | 42294337 | rs2269906 |
| Nalls et al 2019 | FAM171A2 | 17 | 42434630 | rs850738 |
| Nalls et al 2919 | BRIP1 | 17 | 59917366 | rs61169879 |
| Nalls et al 2019 | DNAH17 | 17 | 76425480 | rs666463 |
| Nalls et al 2019 | ASXL3 | 18 | 31304318 | rs1941685 |
| Nalls et al 2019 | MEX3C | 18 | 48683589 | rs8087969 |
| Nalls et al 2019 | CRLS1 | 20 | 6006041 | rs77351827 |
| Nalls et al 2019 | DYRK1A | 21 | 38852361 | rs2248244 |

| | Effect | Reported | OR in | | | | | |
|---|---|---|---|---|---|---|---|---|
| Paper | allele | OR | our study | Direction | P | N | Phet | I |
| Chang et al 2017 | C | 0.92 | 0.878 | same | 3.18E−97 | 5 | 0.156 | 39.77 |
| Chang et al 2017 | C | 1.07 | 1.033 | same | 0.153 | 5 | 0.560 | 0 |
| Chang et al 2017 | T | 0.94 | 0.954 | same | 0.043 | 5 | 0.194 | 34.07 |
| Chang et al 2017 | G | 1.11 | 1.070 | same | 0.178 | 5 | 0.691 | 0 |
| Chang et al 2017 | G | 0.93 | 1.085 | opp | 0.162 | 5 | 0.964 | 0 |
| Chang et al 2017 | G | 0.68 | 0.970 | same | 0.748 | 5 | 0.356 | 8.91 |
| Chang et al 2017 | too rare | | | | | | | |
| Chang et al 2017 | C | 1.15 | 0.998 | opp | 0.968 | 5 | 0.157 | 39.64 |
| Chang et al 2017 | A | 1.12 | 1.077 | same | 7.20E−03 | 5 | 0.137 | 42.67 |
| Chang et al 2017 | too rare | | | | | | | |
| Chang et al 2017 | T | 1.06 | 1.065 | same | 0.030 | 5 | 0.417 | 0 |
| Chang et al 2017 | T | 0.91 | 0.936 | same | 4.58E−03 | 5 | 0.107 | 47.49 |
| Chang et al 2017 | C | 0.93 | 0.097 | same | 0.893 | 5 | 0.177 | 36.68 |
| Chang et al 2017 | T | 1.08 | 1.007 | same | 0.782 | 5 | 0.338 | 11.89 |
| Chang et al 2017 | T | 1.07 | 0.840 | opp | 0.198 | 3 | 0.783 | 0 |
| Chang et al 2017 | T | 1.08 | 1.061 | same | 0.023 | 5 | 0.885 | 0 |
| Chang et al 2017 | C | 0.93 | 0.863 | same | 0.076 | 5 | 0.232 | 28.48 |
| Nalls et al 2014 | too rare | | | | | | | |
| Nalls et al 2014 | T | 1.122 | 1.352 | same | 9.76E−18 | 4 | 0.943 | 0 |
| Nalls et al 2014 | T | 1.131 | 1.031 | same | 0.332 | 5 | 0.071 | 53.69 |
| Nalls et al 2014 | T | 0.875 | 0.929 | same | 0.534 | 3 | 0.584 | 0 |
| Nalls et al 2014 | T | 1.214 | 1.070 | same | 9.63E−03 | 5 | 0.696 | 0 |
| Nalls et al 2014 | too rare | | | | | | | |
| Nalls et al 2014 | T | 1.067 | 1.008 | same | 0.716 | 5 | 0.169 | 37.78 |
| Nalls et al 2014 | A | 0.842 | 0.828 | same | 1.49E−16 | 5 | 0.691 | 0 |
| Nalls et al 2014 | T | 0.786 | 0.859 | same | 7.03E−06 | 5 | 0.774 | 0 |
| Nalls et al 2014 | A | 1.126 | 1.117 | same | 1.53E−06 | 5 | 0.394 | 2.14 |
| Nalls et al 2014 | T | 0.907 | 1.056 | opp | 0.183 | 5 | 0.901 | 0 |
| Nalls et al 2014 | A | 0.76 | 0.703 | same | 7.15E−20 | 4 | 0.192 | 36.68 |
| Nalls et al 2014 | T | 0.826 | 0.897 | same | 0.122 | 4 | 0.232 | 30.01 |
| Nalls et al 2014 | A | 1.11 | 1.067 | same | 0.011 | 5 | 0.573 | 0 |
| Nalls et al 2014 | A | 0.916 | 0.912 | same | 7.59E−05 | 5 | 0.251 | 25.55 |
| Nalls et al 2014 | T | 1.078 | 1.034 | same | 0.078 | 5 | 0.824 | 0 |
| Nalls et al 2014 | too rare | | | | | | | |
| Nalls et al 2014 | too rare | | | | | | | |
| Nalls et al 2014 | A | 0.929 | 0.885 | same | 3.26E−07 | 5 | 0.327 | 13.65 |
| Nalls et al 2014 | T | 1.105 | 1.049 | same | 0.048 | 5 | 0.282 | 20.91 |
| Nalls et al 2014 | T | 1.155 | 0.980 | opp | 0.707 | 5 | 0.091 | 50.19 |
| Nalls et al 2014 | A | 1.105 | 1.093 | same | 1.71E−03 | 5 | 0.871 | 0 |
| Nalls et al 2014 | T | 0.904 | 0.962 | same | 0.090 | 5 | 0.375 | 5.65 |
| Nalls et al 2014 | A | 0.897 | 1.002 | opp | 0.916 | 5 | 0.209 | 31.81 |
| Nalls et al 2014 | A | 1.113 | 1.136 | same | 4.49E−05 | 5 | 0.364 | 7.55 |
| Nalls et al 2014 | A | 1.103 | 1.093 | same | 0.018 | 5 | 0.926 | 0 |
| Nalls et al 2014 | A | 0.939 | 1.032 | opp | 0.329 | 5 | 0.971 | 0 |
| Nalls et al 2014 | too rare | | | | | | | |
| Nalls et at 2014 | A | 0.904 | 0.885 | same | 1.32E−07 | 5 | 0.743 | 0 |
| Nalls et al 2014 | T | 1.097 | 1.043 | same | 0.030 | 5 | 0.300 | 17.94 |
| Nalls et al 2014 | A | 1.111 | 1.036 | same | 0.390 | 3 | 0.605 | 0 |
| Nalls et al 2014 | A | 1.031 | 0.968 | opp | 0.197 | 5 | 0.398 | 1.48 |
| Nalls et al 2019 | C | 1.067 | 1.011 | same | 0.677 | 5 | 0.177 | 36.58 |
| Nalls et al 2019 | T | 0.932 | 1.010 | opp | 0.737 | 5 | 0.847 | 0 |
| Nalls et al 2019 | too rare | | | | | | | |
| Nalls et al 2019 | A | 0.936 | 0.976 | same | 0.395 | 5 | 0.769 | 0 |
| Nalls et al 2019 | T | 1.068 | 0.948 | same | 0.060 | 5 | 0.273 | 22.17 |
| Nalls et al 2019 | T | 1.090 | 1.029 | same | 0.447 | 3 | 0.644 | 0 |
| Nalls et al 2019 | A | 0.939 | 0.938 | same | 0.048 | 5 | 0.278 | 21.42 |
| Nalls et al 2019 | A | 0.940 | 0.980 | same | 0.373 | 5 | 0.257 | 24.62 |
| Nalls et al 2019 | A | 0.919 | 0.918 | same | 8.81E−03 | 5 | 0.311 | 16.32 |
| Nalls et al 2019 | A | 0.938 | 0.902 | same | 0.030 | 5 | 0.655 | 0 |
| Nalls et al 2019 | C | 1.064 | 1.021 | same | 0.373 | 5 | 0.189 | 34.82 |
| Nalls et al 2019 | A | 0.912 | 0.946 | same | 0.036 | 5 | 0.452 | 0 |
| Nalls et al 2019 | T | 0.938 | 0.942 | same | 0.132 | 4 | 0.073 | 56.97 |
| Nalls et al 2019 | T | 1.068 | 1.139 | same | 2.19E−03 | 5 | 0.397 | 1.58 |
| Nalls et al 2019 | A | 1.074 | 1.115 | same | 5.57E−06 | 5 | 0.767 | 0 |
| Nalls et al 2019 | T | 0.802 | 0.803 | same | 4.10E−04 | 5 | 0.461 | 0 |
| Nalls et al 2019 | A | 0.867 | 0.921 | same | 0.011 | 5 | 0.515 | 0 |
| Nalls et al 2019 | T | 0.941 | 0.986 | same | 0.543 | 5 | 0.827 | 0 |
| Nalls et al 2019 | T | 0.940 | 0.907 | same | 1.65E−03 | 5 | 0.354 | 9.26 |
| Nalls et al 2019 | A | 0.924 | 0.959 | same | 0.071 | 5 | 0.905 | 0 |
| Nalls et al 2019 | A | 1.091 | 1.093 | same | 6.01E−03 | 5 | 0.264 | 23.68 |
| Nalls et al 2019 | T | 0.947 | 0.934 | same | 3.41E−03 | 5 | 0.257 | 24.7 |
| Nalls et al 2019 | A | 1.062 | 1.104 | same | 3.28E−03 | 5 | 0.295 | 18.79 |
| Nalls et al 2019 | T | 1.064 | 0.997 | opp | 0.893 | 5 | 0.585 | 0 |
| Nalls et al 2019 | T | 1.070 | 1.003 | same | 0.909 | 5 | 0.248 | 26.04 |
| Nalls et al 2019 | T | 0.948 | 0.970 | same | 0.187 | 5 | 0.394 | 2.14 |
| Nalls et al 2019 | A | 1.074 | 1.079 | same | 6.15E−03 | 5 | 0.030 | 62.66 |
| Nalls et al 2019 | C | 0.937 | 0.867 | same | 0.010 | 4 | 0.568 | 0 |
| Nalls et al 2019 | A | 1.061 | 1.066 | same | 0.026 | 5 | 0.993 | G |
| Nalls et al 2019 | too rare | | | | | | | |
| Nalls et al 2019 | A | 0.945 | 1.042 | opp | 0.124 | 5 | 0.325 | 13.96 |
| Nalls et al 2019 | A | 1.065 | 1.077 | same | 5.30E−03 | 5 | 0.770 | 0 |
| Nalls et al 2019 | A | 0.931 | 1.018 | opp | 0.440 | 5 | 0.992 | 0 |
| Nalls et al 2919 | T | 1.085 | 0.998 | opp | 0.922 | 5 | 0.496 | 0 |
| Nalls et al 2019 | A | 1.079 | 0.947 | opp | 0.365 | 5 | 0.272 | 22.32 |
| Nalls et al 2019 | T | 1.054 | 1.166 | same | 5.90E−05 | 5 | 0.902 | 0 |
| Nalls et al 2019 | T | 0.944 | 1.059 | opp | 0.031 | 5 | 0.865 | 9 |
| Nalls et al 2019 | T | 1.083 | too rare | | | | | |
| Nalls et al 2019 | A | 1.074 | 1.039 | same | 0.103 | 5 | 0.724 | 0 |

*Represented by SNP rs4613239 (G allele) in LD with rs1474955 (r² = 1 in 1000 genomes East Asian).

### EXAMPLE 4

### Replication of Novel Loci in Japanese and European-Ancestry Datasets

To determine if the two novel SNPs are associated with PD risk in other populations, summary statistics from the largest European-ancestry datasets available online, namely the UK Biobank (1,239 cases, 451,025 controls) and the most recent meta-GWAS by the IPDGC (up to 56,306 cases, 1,417,791 controls) was evaluated. Given that the IPDGC dataset includes proxy cases and web-based diagnosed cases and controls, only the subset of clinically diagnosed PD cases consisting of 15,056 cases and 12,637 controls (Table 4) was analysed. In addition, SNPs within these two loci were analysed in 988 cases, 2521 controls from Japan. Both risk variants are present at lower frequencies in European populations compared to Asian populations (Table 4).

Consistent association was observed at SV2C in samples of Japanese (OR=1.11, 95% CI=0.94-1.31, P=0.24) and European-ancestry including IPDGC full (OR=1.07, 95% CI=1.04-1.11; P=3.62×10⁻⁵), and IPDGC clinically-diagnosed sub-dataset (OR=1.13, 95% CI=1.06-1.21; P=2.95×10⁻⁴) and UK Biobank data (OR=1.09, 95% CI=0.94-1.26; P=0.25). Based on the full replication datasets, significant replication was observed at the SV2C locus (OR_{replication meta-analysis}=1.07; 95% CI=1.04-1.11; P_{replication meta-analysis}=9.74×10⁻⁶; I²=0%, Pₕₑₜ=0.92; OR_{combined meta-analysis}=1.10; 95% CI=1.07-1.13; P_{combined meta-analysis}=6.02×10⁻¹⁰; I²=48%, Pₕₑₜ=0.06) (Table 4). Meta-analysis of Asian consortium discovery samples with the European and Japanese clinically-diagnosed PD replication samples provided strong support for the association at both the lead SNP SV2C rs246814 (OR=1.16; 95% CI=1.11-1.21; P=1.17×10⁻¹⁰; I²=0%. Pₕₑₜ=0.50) (Table 4) and the missense variant p.Asp543Asn rs31244 (OR=1.16; 95% CI=1.11-1.21; P=1.80×10⁻¹⁰; I²=0%. Pₕₑₜ=0.53) with low inter-cohort and inter-ethnic heterogeneity.

The WBSCR17 SNP rs9638616 did not appear to be associated with PD risk in European data, in IPDGC full (OR=1.00, 95% CI=0.98-1.02; P=0.76) and clinically-diagnosed datasets (OR=1.01, 95% CI=0.95-1.06; P=0.85), UK BioBank (OR=0.97, 95% CI=0.89-1.06; P=0.53) or Japan (OR=1.04, 95% CI=0.94-1.16; P=0.43) PD GWAS. This SNP (OR=1.06; 95% CI=1.03-1.10; P=8.37×10⁻⁵; I²=67.1%; Pₕₑₜ=3.40×10⁻³) and locus did not reach genome-wide significance in a meta-analysis between the discovery, Japanese and European clinically-diagnosed PD samples (Table 4).

### EXAMPLE 5

### Polygenic Risk Score Modeling

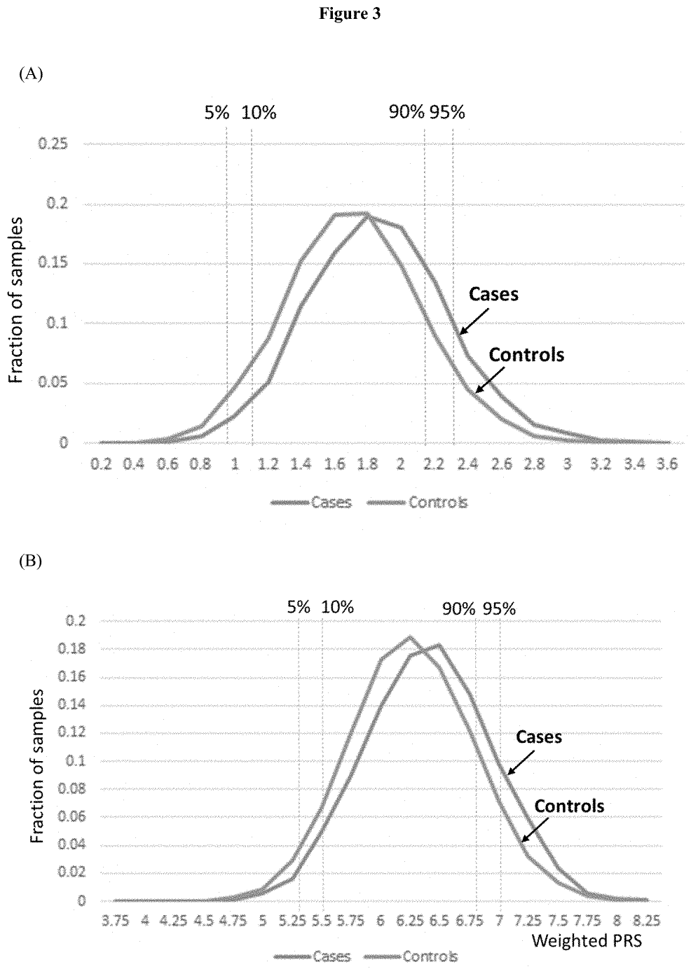

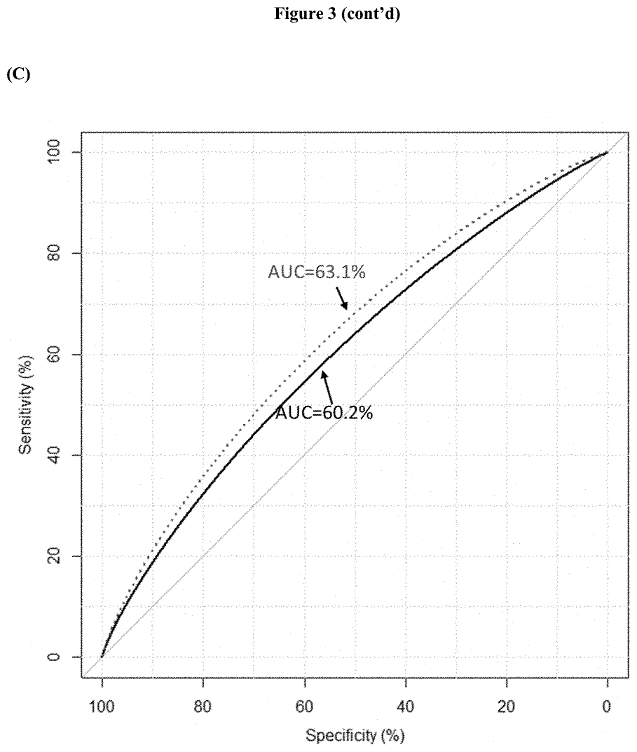

PRS was calculated based on the 11 genome-wide significant SNPs identified in this Asian PD study (Table 1 and 7). To evaluate the utility of SNPs identified by European GWAS in predicting risk in the Asian population, separate scores were calculated using 90 risk variants (78 polymorphic) from previously-reported European loci using effect sizes derived from the GWAS in which they were first reported. The PRS distribution was then evaluated in the largest Asian subset of 2,536 PD cases and 21,840 controls from Singapore and Malaysia (FIG. 3).

In the weighted PRS distribution based on the 11 Asian SNPs, a 4.0- and 3.5-fold difference was observed in risk between the top and bottom 5% and 10% of the PRS distribution in controls (FIG. 3A) respectively. It was also observed that higher PRS scores are significantly correlated with a younger age of onset in PD patients (β=−1.784, P=5.17×10⁻⁴), consistent with previous observations.¹² In contrast, there was no correlation between age of controls and PRS (β=0.16, P=0.21). A 0.29-year decrease in age of onset was estimated for every additional copy of risk allele present among the 11 loci. Assessment within the present Asian PD dataset of weighted PRS scores based on the 78 European SNPs revealed a 2.9- and 2.2-fold difference in risk between the top and bottom 5% and 10% of PRS distribution in controls respectively (FIG. 3B).

These 11 Asian SNPs were estimated to account for about 2.61% of the variance in PD risk in this dataset (AUC=60.4%; 95% CI=59.5-61.8%), while the 78 polymorphic European SNPs explained about 2.57% of the variance in the same dataset (AUC=60.2%; 95% CI=59.0-61.2%). The AUCs were not significantly different between the two models (P=0.825). While the European PD SNPs are still able to discriminate Asian cases and controls, their utility is limited by allelic heterogeneity, LD differences and variability in effect sizes because of gene-gene or gene-environment interactions. Combining the European and Asian loci (Table 8), a significant improvement was observed in AUC (63.1%; 95% CI: 62.1-64.4%) over the model based on European loci alone (P=6.81×10⁻¹²) (FIG. 3C), and similar to that in European samples (AUC=65.1%). Similar improvements were observed in the China (66.2% vs 64.7%; P=0.005) and South Korean (69.5% vs 68.0%; P=0.036) datasets. These analyses suggest that the data resolution conferred by PRS modelling will progressively improve as further research in Asian samples reveal additional PD risk loci.

TABLE 7
List of 11 SNPs in the Asian PD study

| | | | | Effect | Reported | Genetic | |
|---|---|---|---|---|---|---|---|
| CHR | BP | SNP | Paper | allele | effect size | locus | Remarks |
| 4 | 90,682,474 | rs6826785 | The present invention | C | 0.292 | SNCA | |
| 12 | 40,387,749 | rs141336855 | The present invention | T | 0.689 | LRRK2 | |
| 1 | 205,756,484 | rs6679073 | The present invention | A | 0.215 | PARK16 | |
| 3 | 182,735,211 | rs2292056 | The present invention | G | −0.192 | MCCC1 | |
| 1 | 226,846,712 | rs16846351 | The present invention | G | 0.285 | ITPKB | |
| 4 | 77,101,068 | rs3816248 | The present invention | C | −0.135 | FAM47E-SCARB2 | |
| 11 | 83,510,117 | rs12278023 | The present invention | C | −0.129 | DLG2 | |
| 7 | 70,750,493 | rs9638616 | The present invention | T | 0.128 | WBSCR17 | new |
| 6 | 112,151,452 | rs1887316 | The present invention | A | −0.192 | FYN | |
| 5 | 75,599,208 | rs246814 | The present invention | T | 0.217 | SV2C | new, 2 SNPs from same |
| 5 | 75,594,743 | rs31244 | The present invention | G | 0.211 | SV2C | locus, only need 1 |
| 18 | 40,678,235 | rs4130047 | The present invention | C | 0.127 | RIT2 | |

Table 8 List of SNPs for PRS

| | | | | Effect | Reported |
|---|---|---|---|---|---|
| CHR | BP | SNP | Paper | allele | effect size |
| 4 | 90,682,474 | rs6826785 | The present invention | C | 0.292 |
| 12 | 40,387,749 | rs141336855 | The present invention | T | 0.689 |
| 1 | 205,756,484 | rs6679073 | The present invention | A | 0.215 |
| 3 | 182,735,211 | rs2292056 | The present invention | G | −0.192 |
| 1 | 226,846,712 | rs16846351 | The present invention | G | 0.285 |
| 4 | 77,101,068 | rs3816248 | The present invention | C | −0.135 |
| 11 | 83,510,117 | rs12278023 | The present invention | C | −0.129 |
| 7 | 70,750,493 | rs9638616 | The present invention | T | 0.128 |
| 6 | 112,151,452 | rs1887316 | The present invention | A | −0.192 |
| 5 | 75,599,208 | rs246814 | The present invention | T | 0.217 |
| 5 | 75,594,743 | rs31244 | The present invention | G | 0.211 |
| 18 | 40,678,235 | rs4130047 | The present invention | C | 0.127 |
| 12 | 130,933,889 | rs12305875 | The present invention | T | −0.102 |
| 12 | 40,713,845 | rs33949390 | Asian LRRK2 risk variant_R1628P | C | ~0.693 |
| 12 | 40,757,328 | rs34778348 | Asian LRRK2 risk variant_G2385R | A | ~0.693 |
| 12 | 40,713,901 | rs11564148 | Asian LRRK2 risk variant_S1647T | A | ~0.693 |
| 1 | 155,205,043 | rs421016 | Asian GBA risk variant_L444P | G | >0.693 |
| 1 | 155,208,006 | rs364897 | Asian GBA risk variant_N188S | C | >0.693 |
| 1 | 155,135,036 | rs35749011 | Nalls et al 2014 | A | 0.601 |
| 1 | 205,723,572 | rs823118 | Nalls et al 2014 | T | 0.115 |
| 1 | 232,664,611 | rs10797576 | Nalls et al 2014 | T | 0.123 |
| 2 | 135,539,967 | rs6430538 | Nalls et al 2014 | T | −0.134 |
| 2 | 169,110,394 | rs1474055 | Nalls et al 2014 | T | 0.194 |
| 3 | 87,520,857 | rs115185635 | Nalls et al 2014 | C | 0.133 |
| 3 | 160,992,864 | rs34016896 | Nalls et al 2014 | T | 0.065 |
| 3 | 182,762,437 | rs12637471 | Nalls et al 2014 | A | −0.172 |
| 4 | 951,947 | rs34311866 | Nalls et al 2014 | T | −0.241 |
| 4 | 15,737,101 | rs11724635 | Nalls et al 2014 | A | 0.119 |
| 4 | 77,198,986 | rs6812193 | Nalls et al 2014 | T | −0.098 |
| 4 | 90,626,111 | rs356182 | Nalls et al 2014 | A | −0.274 |
| 6 | 32,666,660 | rs9275326 | Nalls et al 2014 | T | −0.191 |
| 7 | 23,293,746 | rs199347 | Nalls et al 2014 | A | 0.104 |
| 8 | 16,697,091 | rs591323 | Nalls et al 2014 | A | −0.088 |
| 8 | 89,373,041 | rs60298754 | Nalls et al 2014 | T | 0.075 |
| 10 | 15,561,543 | rs7077361 | Nalls et al 2014 | T | 0.088 |
| 10 | 121,536,327 | rs117896735 | Nalls et al 2014 | A | 0.485 |
| 11 | 83,544,472 | rs3793947 | Nalls et al 2014 | A | −0.074 |
| 11 | 133,765,367 | rs329648 | Nalls et al 2014 | T | 0.100 |
| 12 | 40,614,434 | rs76904798 | Nalls et al 2014 | T | 0.144 |
| 12 | 123,303,586 | rs11060180 | Nalls et al 2014 | A | 0.100 |
| 14 | 55,348,869 | rs11158026 | Nalls et al 2014 | T | −0.101 |
| 14 | 67,984,370 | rs1555399 | Nalls et al 2014 | A | −0.109 |
| 15 | 61,994,134 | rs2414739 | Nalls et al 2014 | A | 0.107 |
| 16 | 31,121,793 | rs14235 | Nalls et al 2014 | A | 0.098 |
| 17 | 17,715,101 | rs11868035 | Nalls et al 2014 | A | −0.063 |
| 17 | 43,994,648 | rs17649553 | Nalls et al 2014 | T | −0.263 |
| 18 | 40,673,380 | rs12456492 | Nalls et al 2014 | A | −0.101 |
| 19 | 2,363,319 | rs62120679 | Nalls et al 2014 | T | 0.093 |
| 20 | 3,168,166 | rs8118008 | Nalls et al 2014 | A | 0.105 |
| 21 | 16,914,905 | rs2823357 | Nalls et al 2014 | A | 0.031 |
| 1 | 226,916,078 | rs4653767 | Chang et al 2017 | C | −0.083 |
| 2 | 102,413,116 | rs34043159 | Chang et al 2017 | C | 0.068 |
| 2 | 166,133,632 | rs353116 | Chang et al 2017 | T | −0.062 |
| 3 | 18,277,488 | rs4073221 | Chang et al 2017 | G | 0.104 |
| 3 | 48,748,989 | rs12497850 | Chang et al 2017 | G | −0.073 |
| 3 | 52,816,840 | rs143918452 | Chang et al 2017 | G | −0.386 |
| 4 | 114,360,372 | rs78738012 | Chang et al 2017 | C | 0.131 |
| 5 | 60,273,923 | rs2694528 | Chang et al 2017 | C | 0.140 |
| 6 | 27,681,215 | rs9468199 | Chang et al 2017 | A | 0.113 |
| 8 | 11,707,174 | rs2740594 | Chang et al 2017 | A | 0.095 |
| 8 | 22,525,980 | rs2280104 | Chang et al 2017 | T | 0.058 |
| 9 | 17,579,690 | rs13294100 | Chang et al 2017 | T | −0.094 |
| 10 | 15,569,598 | rs10906923 | Chang et al 2017 | C | −0.073 |
| 14 | 88,472,612 | rs8005172 | Chang et al 2017 | T | 0.077 |
| 16 | 19,279,464 | rs11343 | Chang et al 2017 | T | 0.068 |
| 16 | 52,599,188 | rs4784227 | Chang et al 2017 | T | 0.077 |
| 17 | 40,698,158 | rs601999 | Chang et al 2017 | C | −0.073 |
| 1 | 161,469,054 | rs6658353 | Nalls et al 2019 | C | 0.065 |
| 1 | 171,719,769 | rs11578699 | Nalls et al 2019 | T | −0.070 |
| 2 | 18,147,848 | rs76116224 | Nalls et al 2019 | A | 0.110 |
| 2 | 96,000,943 | rs2042477 | Nalls et al 2019 | A | −0.066 |
| 3 | 28,705,690 | rs6808178 | Nalls et al 2019 | T | 0.066 |
| 3 | 122,196,892 | rs55961674 | Nalls et al 2019 | T | 0.086 |
| 3 | 151,108,965 | rs11707416 | Nalls et al 2019 | A | −0.063 |
| 3 | 161,077,630 | rs1450522 | Nalls et al 2019 | A | −0.062 |
| 4 | 17,968,811 | rs34025766 | Nalls et al 2019 | A | −0.084 |
| 4 | 170,583,157 | rs62333164 | Nalls et al 2019 | A | −0.064 |
| 5 | 102,365,794 | rs26431 | Nalls et al 2019 | C | 0.062 |
| 5 | 134,199,105 | rs11950533 | Nalls et al 2019 | A | −0.092 |
| 6 | 30,108,683 | rs9261484 | Nalls et al 2019 | T | −0.064 |
| 6 | 72,487,762 | rs12528068 | Nalls et al 2019 | T | 0.066 |
| 6 | 112,243,291 | rs997368 | Nalls et al 2019 | A | 0.071 |
| 6 | 133,210,361 | rs75859381 | Nalls et al 2019 | T | −0.221 |
| 7 | 66,009,851 | rs76949143 | Nalls et al 2019 | A | −0.143 |
| 8 | 130,901,909 | rs2086641 | Nalls et al 2019 | T | −0.061 |
| 9 | 34,046,391 | rs6476434 | Nalls et al 2019 | T | −0.062 |
| 10 | 104,015,279 | rs10748818 | Nalls et al 2019 | A | −0.079 |
| 11 | 10,558,777 | rs7938782 | Nalls et al 2019 | A | 0.087 |
| 12 | 46,419,086 | rs7134559 | Nalls et al 2019 | T | −0.054 |
| 12 | 133,063,768 | rs11610045 | Nalls et al 2019 | A | 0.060 |
| 13 | 49,927,732 | rs9568188 | Nalls et al 2019 | T | 0.062 |
| 13 | 97,865,021 | rs4771268 | Nalls et al 2019 | T | 0.068 |
| 14 | 37,989,270 | rs12147950 | Nalls et al 2019 | T | −0.053 |
| 14 | 75,373,034 | rs3742785 | Nalls et al 2019 | A | 0.071 |
| 16 | 28,944,396 | rs2904880 | Nalls et al 2019 | C | −0.065 |
| 16 | 50,736,656 | rs6500328 | Nalls et al 2019 | A | 0.059 |
| 16 | 58,587,672 | rs200564078 | Nalls et al 2019 | T | 0.859 |
| 17 | 7,355,621 | rs12600861 | Nalls et al 2019 | A | −0.057 |
| 17 | 42,294,337 | rs2269906 | Nalls et al 2019 | A | 0.063 |
| 17 | 42,434,630 | rs850738 | Nalls et al 2019 | A | −0.071 |
| 17 | 59,917,366 | rs61169879 | Nalls et al 2019 | T | 0.082 |
| 17 | 76,425,480 | rs666463 | Nalls et al 2019 | A | 0.076 |
| 18 | 31,304,318 | rs1941685 | Nalls et al 2019 | T | 0.053 |
| 18 | 48,683,589 | rs8087969 | Nalls et al 2019 | T | −0.058 |
| 20 | 6,006,041 | rs77351827 | Nalls et al 2019 | T | 0.080 |
| 21 | 38,852,361 | rs2248244 | Nalls et al 2019 | A | 0.071 |

Discussion

The largest multicenter Asian GWAS on PD to date has been conducted, analysing 31,575 subjects (6,724 cases, 24,851 controls) from six regions across East Asia. Genome-wide significant association signals were observed at 11 loci and consistent association at nominal significance (P<0.05) at 51 other previously-reported loci. Of the two novel loci identified, strong replication of the association at SV2C was observed across three independent sample collections from European-ancestry and Japanese populations.

The top-associated haplotype at SV2C is consistent between Asian and European-ancestry samples. Despite differences in LD patterns, the top SNP rs246814 is in near perfect LD with p.Asp543Asn (rs31244) and two other flanking SNPs rs246813 and rs246815 in both Asians and Europeans, suggesting that the functional variant likely resides on this common haplotype. The lack of significant replication at WBSCR17 in the Japanese dataset may be attributed to the small effect sizes observed at this locus (68.5% power to detect an association at alpha=0.05). There is no significant genetic heterogeneity between the Japanese replication samples and the present East Asian discovery GWAS samples (Pₕₑₜ=0.24, I²=25.6%).

This study is notable in several aspects. Firstly, strong evidence is provided for the association of genetic variants (including a non-synonymous variant) in SV2C with PD risk in humans. The strong association reported now between this naturally occurring SV2C missense allele and increased risk of PD lends credence to SV2C being a potential therapeutic target.

In addition, the present results demonstrate that there are significant differences in the overall underlying genetic architecture, involving allele frequency and LD patterns and allelic heterogeneity between Europeans and Asians, leading to an improvement in the PRS model upon inclusion of SNPs identified in Asians.

### REFERENCES

Chang D, Nails M A, Hallgrimsdottir I B, et al. A meta-analysis of genome-wide association studies identifies 17 new Parkinson's disease risk loci. Nat Genet 2017; 49(10): 1511-6.
Nails M A, Pankratz N, Lill C M, et al. Large-scale meta-analysis of genome-wide association data identifies six new risk loci for Parkinson's disease. Nat Genet 2014; 46(9): 989-93.
Nails M A, Blauwendraat C, Vallerga C L, et al. Identification of novel risk loci, causal insights, and heritable risk for Parkinson's disease: a meta-analysis of genome-wide association studies. Lancet Neurol 2019; 18(12): 1091-102.

Equivalents

The foregoing examples are presented for the purpose of illustrating the invention and should not be construed as imposing any limitation on the scope of the invention. It will readily be apparent that numerous modifications and alterations may be made to the specific embodiments of the invention described above and illustrated in the examples without departing from the principles underlying the invention. All such modifications and alterations are intended to be embraced by this application.

1. A method of identifying whether a subject is at risk of developing Parkinson's disease (PD), whether a subject is suffering from PD, whether a subject is in need of early therapeutic intervention for PD, (ii) determining a prognosis of a subject with PD or a subject at risk of developing PD, or (iii) calculating a polygenic risk score (PRS) of a subject of developing PD, the method comprising:
a. obtaining a DNA sample from the subject; and
b. detecting the presence of a genetic variant at the loci of one or more genes selected from the group consisting of SV2C, WBSCR17, PARK16, ITPKB, MCCC1, SNCA, FAM47E-SCARB2, FYN, DLG2, LRRK2, RIT2, and combinations thereof in the sample;
wherein the presence of one or more genetic variants identifies that the subject is at risk of developing PD, the subject is suffering from PD, the subject is in need of early therapeutic intervention for PD, or indicates that the subject has a poor prognosis,
wherein the method further comprises:
c. measuring a total number of the genetic variants detected in step b to calculate a PRS of a subject of developing PD.

2. (canceled)

3. The method of claim 1 wherein the method further comprises detecting the presence of a genetic variant at the loci of one or more genes selected from ILIR2, SCN3A, SATB1, NCKIPSD, CDC71, ALAS1, TLR9, DNAH1, BAP1, PHF7, NISCH, STAB1, ITIH3, ITIH4, ANK2, CAMK2D, ELOVL7, ZNF184, CTSB, SORBS3, PDLIM2, C8orf58, BIN3, SH3GL2, FAM171A1, GALC, COQ7, TOX3, ATP6V0A1, PSMC3I, TUBG2, GBA-SYT11, RAB7L1-NUCKS1, SIPA1L2, ACMSD-TMEM163, STK39, KRT8P25-APOOP2, NMD3, TMEM175-GAK-DGKQ, BST1, HLA-DQB1, GPNMB, FGF20, MMP16, ITGA8, INPP5F, MIR4697, LRRK2, CCDC62, GCH1, TMEM229B, VPS13C, BCKDK-STX1B, SREBF1-RAI1, MAPT, SPPL2B, DDRGK1, USP25, FCGR2A, VAMP4, KCNS3, KCNIP3, LINC00693, KPNA1, MED12L, SPTSSB, LCORL, CLCN3, PAM, C5orf24, TRIM40, RIMS1, RPS12, GS1-124K5.11, FAM49B, UBAP2, GBF1, RNF141, SCAF11, FBRSL1, CAB39L, MBNL2, MIPOL1, RPS6KL1, CD19, NOD2, CNOT1, CHRNB1, UBTF, FAM171A2, BRIP1, DNAH17, ASXL3, MEX3C, CRLS1, DYRK1A, and combinations thereof.

4.-5. (canceled)

6. The method of claim 1, wherein the total number of genetic variants is weighted by an effect size of each variant.

7. The method of claim 1, wherein the PRS of the subject is compared with PRSs in a reference population to determine a percentile risk of the subject's risk of developing PD.

8. The method of claim 7, wherein a subject with a higher percentile PRS has a higher risk of developing PD compared to a subject with a lower percentile PRS.

9. The method of claim 1, wherein the one or more genetic variants is a polymorphism.

10. The method of claim 9, wherein the polymorphism is a single nucleotide polymorphism (SNP) or single nucleotide variant (SNV).

11. The method of claim 10, wherein the genetic variant is an effect allele or risk allele of the SNP or SNV.

12. The method of claim 1, wherein the genetic variant is a single nucleotide polymorphism (SNP) selected from the group consisting of rs6826785, rs141336855, rs6679073, rs2292056, rs16846351, rs3816248, rs12278023, rs9638616, rs1887316, rs246814, rs31244, rs4130047, and combinations thereof.

13. The method of claim 12, wherein an effect allele of rs6826785 is cytosine (C), an effect allele of rs141336855 is thymine (T), an effect allele of rs6679073 is adenine (A), an effect allele of rs2292056 is guanine (G), an effect allele of rs16846351 is guanine (G), an effect allele of rs3816248 is cytosine (C), an effect allele of rs12278023 is cytosine (C), an effect allele of rs9638616 is thymine (T), an effect allele of rs1887316 is adenine (A), an effect allele of rs246814 is thymine (T), an effect allele of rs31244 is guanine (G), and an effect allele of rs4130047 is cytosine (C).

14. The method of claim 3, wherein the genetic variant is a single nucleotide polymorphism (SNP) selected from the group consisting of rs34043159, GSA-rs353116, rs4073221, rs12497850, rs143918452, rs78738012, rs2694528, rs9468199, rs2740594, rs2280104, rs13294100, rs10906923, rs8005172, rs11343, rs4784227, rs601999, rs35749011, rs10797576, rs6430538, rs1474055, rs115185635, rs34016896, rs34311866, rs11724635, rs9275326, rs199347, rs591323, rs60298754, rs7077361, rs117896735, rs329648, rs11060180, rs11158026, rs1555399, rs2414739, rs14235, rs11868035, rs17649553, rs113579895, rs62120679, rs8118008, rs2823357, rs6658353, rs11578699, rs76116224, rs2042477, rs6808178, rs55961674, rs11707416, rs1450522, rs34025766, rs62333164, rs26431, rs11950533, rs9261484, rs12528068, rs75859381, rs76949143, rs2086641, rs6476434, rs10748818, rs7938782, rs7134559, GSA-rs11610045, rs9568188, rs4771268, rs12147950, rs3742785, rs2904880, rs6500328, rs200564078, rs12600861, rs2269906, rs850738, rs61169879, rs666463, rs1941685, rs8087969, rs77351827, rs2248244, rs4613239, rs1474055, and combinations thereof.

15. The method of claim 1, wherein the subject is of Asian ethnicity or ancestry.

16. The method of claim 15, wherein the subject is of Han Chinese ancestry or Chinese ethnicity or ancestry with no mixed ancestry, or a South Korean ethnicity or ancestry.

17. A kit comprising one or more reagents to detect the presence of a genetic variant at the loci of one or more genes selected from the group consisting of SV2C ,WBSCR17, PARK16, ITPKB, MCCC1, SNCA, FAM47E-SCARB2, FYN, DLG2, LRRK2, RIT2, and combinations thereof in a sample, together with instructions for use.

18. The kit of claim 17, further comprising reagents to detect the presence of a genetic variant at loci of one or more genes selected from the group consisting of ILIR2, SCN3A, SATB1, NCKIPSD, CDC71, ALAS1, TLR9, DNAH1, BAP1, PHF7, NISCH, STAB1, ITIH3, ITIH4, ANK2, CAMK2D, ELOVL7, ZNF184, CTSB, SORBS3, PDLIM2, C8orf58, BIN3, SH3GL2, FAM171A1, GALC, COQ7, TOX3, ATP6V0A1, PSMC3I, TUBG2, GBA-SYT11, RAB7L1-NUCKS1, SIPA1L2, ACMSD-TMEM163, STK39, KRT8P25-APOOP2, NMD3, TMEM175-GAK-DGKQ, BST1, HLA-DQB1, GPNMB, FGF20, MMP16, ITGA8, INPP5F, MIR4697, LRRK2, CCDC62, GCH1, TMEM229B, VPS13C, BCKDK-STXIB, SREBF1-RAI1, MAPT, SPPL2B, DDRGK1, USP25, FCGR2A, VAMP4, KCNS3, KCNIP3, LINC00693, KPNA1, MED12L, SPTSSB, LCORL, CLCN3, PAM, C5orf24, TRIM40, RIMS1, RPS12, GS1-124K5.11, FAM49B, UBAP2, GBF1, RNF141, SCAF11, FBRSL1, CAB39L, MBNL2, MIPOL1, RPS6KL1, CD19, NOD2, CNOT1, CHRNB1, UBTF, FAM171A2, BRIP1, DNAH17, ASXL3, MEX3C, CRLS1, DYRK1A, and combinations thereof.

19. The kit of claim 17, wherein the one or more reagents comprises a reagent to isolate a nucleic acid from the sample and at least one primer and/or at least one probe for amplification of a sequence encoding the genetic variant or part thereof.

20. The kit of claim 17 for identifying whether a subject is at risk of developing Parkinson's Disease (PD), whether a subject is suffering from PD, whether a subject is in need of early therapeutic intervention for PD, determining the prognosis of a subject with PD or a subject at risk of developing PD, calculating a PRS of a subject of developing PD, or combinations thereof.

21. A PD biomarker, wherein the biomarker is a genetic variant at loci of one or more genes selected from the group consisting of SV2C, WBSCR17, PARK16, ITPKB, MCCC1, SNCA, FAM47E-SCARB2, FYN, DLG2, LRRK2, RIT2, and combinations thereof.
